(19) United States
(12) Patent Application Publication
(10) Pub. No.: US 2024/0117292 A1
(43) Pub. Date: Apr. 11, 2024

(54) 3D BIOPHOTONIC DEVICES FOR OPTICAL ELECTROPHYSIOLOGY AND METHODS OF USE THEREOF

(71) Applicant: THE GEORGE WASHINGTON UNIVERSITY, Washington, DC (US)

(72) Inventors: Emilia ENTCHEVA, Washington, DC (US); Christianne Joan CHUA, Washington, DC (US)

(21) Appl. No.: 18/509,951

(22) Filed: Nov. 15, 2023

Related U.S. Application Data
(63) Continuation of application No. PCT/US22/29848, filed on May 18, 2022.
(60) Provisional application No. 63/190,178, filed on May 18, 2021.

(51) Int. Cl. C12M 1/42; C12N 5/077

(52) U.S. Cl. CPC C12M 35/02; C12M 35/04; C12M 35/06; C12N 5/0657; A61N 1/362

(57) ABSTRACT

Embodiments of the instant disclosure relate to compositions and methods of use thereof that allow for faster integration of acute cardiac pacing experiments compared to the direct genetic modification methods currently in use. Embodiments provided herein provide for spheroids that may be stored, transported, and deployed on site to confer optical pacing of cardiac tissue for use in high-throughput functional in vitro screening assays.

### CROSS-REFERENCE TO RELATED APPLICATIONS

This application is a continuation of PCT Application No. PCT/US2022/029848, filed May 18, 2022, for 3D BIOPHOTONIC DEVICES FOR OPTICAL ELECTROPHYSIOLOGY AND METHODS OF USE THEREOF, which claims the benefit of U.S. Provisional Application No. 63/190,178, filed on May 18, 2021, the disclosures of which are hereby incorporated by reference in their entireties.

### STATEMENT REGARDING FEDERALLY SPONSORED RESEARCH

This invention was made with government support under Grant No. R01 HL144157 awarded by the National Institutes for Health, Grant No. EFMA 1830941 awarded by the National Science Foundation, and Grant No. PFI 1827535 awarded by the National Science Foundation. The government has certain rights in the invention.

### INCORPORATION BY REFERENCE OF SEQUENCE LISTING PROVIDED ELECTRONICALLY

This application contains a substitute Sequence Listing submitted as an electronic XML file”, the contents of which are incorporated by reference in their entirety. The electronic XML file is 7 kilobytes (KB) in size, was created on 13 Dec. 2023, and is titled “GW085 (091019-690976)—Sequence Listing”.

### FIELD

Embodiments of the instant disclosure relate to novel methods and compositions for faster integration of acute cardiac pacing experiments. In certain embodiments, spheroids prepared according to the methods disclosed herein may be stored, transported, and deployed on site to confer optical pacing of cardiac tissue for use in high-throughput functional screening assays.

### BACKGROUND

The development of new drugs is lengthy and inefficient, where the approval process alone takes on average 7-10 years. In the United States, <0.05% of all compounds undergoing preclinical tests become marketed drugs, and <30% of compounds evaluated in clinical trials make it to market. Perhaps most costly, and with greatest negative societal and ethical impact, is the withdrawal of drugs from the market after they have been approved. Insufficient or inadequate tools for predicting failure before more expensive phases of testing both in animal and human, drive up the drug costs and decrease the desire for pharmaceutical companies to pursue more ‘high-risk’ drugs that would result in little payout. Ultimately, developing tools for improved failure prediction of a drug in earlier stages of the development process is necessary to reverse current trends. In the last 40 years, over 20% of drugs discontinued at all phases of development, including discovery, preclinical and clinical evaluation, and post-market surveillance has been due to cardiac toxicity, where unintended interactions with cardiac ion channels result in pro-arrhythmic effects. In response, international regulatory agreements were developed that mandate testing of all new drugs, both cardiac and non-cardiac, for cardiac liability. As such, there is a need in the field for new compositions that can be integrated in a robotics-amenable workflow to study human cardiac electrophysiology and drug action in a rigorous, scalable, and high-throughput manner.

### SUMMARY

The present disclosure is based, at least in part, on the discovery of “spark-cell” spheroids that are usable as a “reagent” which may eventually be stored, transported and deployed on site to confer optical pacing of cardiac tissue.

Aspects of the present disclosure provide for cell-stimulating device compositions. In certain embodiments, a cell-stimulating device disclosed herein may comprising a tissue structure which can comprise: a plurality of cell bodies selected from at least one of biological cell bodies or non-biological cell bodies; and disposed in the cell bodies at least one of an entity capable of translating one or more external stimuli into a local electric field change or a combination of a first entity capable of translating one or more external stimuli into a local electric field change and a second entity capable of converting photons having a first energy to photons having a second energy.

In some embodiments, the plurality of cell bodies may comprise about 100 to about 10,000 cell bodies. In some embodiments, the tissue structure can have a diameter of between 0.15 mm and 1 mm.

In some embodiments, the one or more external stimuli may be selected from the group consisting of electrical stimulation, optical stimulation, mechanical stimulation and magnetic stimulation. In some aspects, a cell-stimulating device disclosed herein may respond to one or more external stimuli that is electrical stimulation. In some embodiments, a cell-stimulating device disclosed herein may comprise an ion-channel capable of causing electrical spiking. In some aspects, ion-channel capable of causing electrical spiking can be Kir2.1, Nav1.5, or both. In some embodiments, a cell-stimulating device disclosed herein may respond to one or more external stimuli that is optical stimulation.

In some embodiments, a cell-stimulating device disclosed herein may have an entity disposed in the cell bodies wherein the entity is a light-gated ion channel or a light-sensitive nanoparticle, such as a plasmonic nanoparticle. In some aspects, the ion channel may be an opsin. In some other aspects, the opsin may be Channelrhodopsin-2.

In some embodiments, a cell-stimulating device disclosed herein may respond to one or more external stimuli that is mechanical stimulation. In some aspects, cell bodies may have an entity disposed in the body, wherein the entity can be a mechanosensitive ion channel entity. In some other aspects, the mechanosensitive ion channel entity can be Piezol. In some embodiments, a cell-stimulating device disclosed herein may respond to a mechanical stimulation delivered by ultrasound, fluid flow, pressure puffs, or any combination thereof

In some embodiments, the one or more external stimuli can be a combination of magnetic stimulation and one of the group consisting of electrical stimulation, optical stimulation and mechanical stimulation. In some aspects, the entity disposed in the cell bodies can be a magnetic nanoparticle. In some other aspects, the magnetic nanoparticle may be about 200 nm diameter dextran iron-oxide composite. In some other aspects, the entity disposed in the cell bodies can be a magnetic microparticle. In some aspects, magnetic microparticle may have a diameter up to about 500 μm.

In some embodiments, a cell-stimulating device disclosed herein may have a second entity configured to emit photons in the absence of external photons and wherein the emitted photons can activate the first entity to induce a local electric field change. In some aspects, the tissue structure has a spheroid shape. In some aspects, the tissue structure has previously been subjected to cryopreservation. In some embodiments, the cryopreservation was performed when the tissue structure had a diameter of between about 100 μm and about 300 μm. In some aspects, the tissue structure had a diameter of 200 μm.

In some embodiments, a cell-stimulating device disclosed herein may have been subjected to cryopreservation that was performed in freezing medium comprising 10% dimethyl sulfoxide (DMSO), 30% Dulbecco's Modified Eagle's Medium (DMEM), and 60% Fetal Bovine Serum (FBS). In some embodiments, a cell-stimulating device disclosed herein may have a tissue structure that was grown in an ultra-low-adhesion microplate.

In some embodiments, the cryopreservation was performed with a controlled cooling rate of 1 degree Celsius per minute overnight in a negative 80 degree Celsius freezer. In some other embodiments, the cryopreservation was additionally performed with transfer from the negative 80 degree Celsius freezer to liquid nitrogen after the controlled cooling overnight. In some aspects, after cryopreservation, the tissue structure was then quickly thawed in a 37 degree Celsius water bath. In some other aspects, a cell-stimulating device disclosed herein may, within 5 minutes from insertion in the water bath, have ice crystals were mechanically removed from the freezing medium.

In some embodiments, a cell-stimulating device disclosed herein may have its tissue structure first transferred to a culture medium after thawing wherein the culture medium comprises the mixture 98% Dulbecco's Modified Eagle's Medium (DMEM) and 2% Fetal Bovine Serum (FBS) and transferred to an ultra-low-adhesion microplate. In some aspects, the tissue structure was further cultured for at least 4 days after thawing before being utilized to stimulate cells.

Aspects of the present disclosure also provide for systems for studying the effect of a local electric field change on cells. In certain embodiments, systems for studying the effect of a local electric field change on cells may comprise: a cell-stimulating device disclosed herein; one or more excitable cells that are responsive to a local electric field change; and, a dye or opsin capable of emitting light of a first wavelength in response to an electrical change in the excitable cell and or a dye capable of emitting light of a second wavelength in response to a change in cytoplasmic calcium concentration in the excitable cell. In some aspects, the cell-stimulating device and the excitable cell may be in the same microwell of a multi-well microplate. In some other aspects, the cell-stimulating device and one or more of the excitable cells may be in contact.

In some embodiments, systems disclosed herein may further comprise a magnet or electromagnet and wherein the timing of said contact can be controlled rapidly via a magnetic field. In some embodiments, systems disclosed herein may further comprise a light source capable of stimulating the cell-stimulating device and a sensor capable of sensing photons that have been converted from a first energy to a second energy. In some embodiments, systems disclosed herein may further comprise a means for providing to the cell-stimulating device a mechanical stimulus selected from the group consisting of ultrasound, fluid flow, and pressure puffs. In some embodiments, systems disclosed herein may further comprise a light source capable of stimulating the cell-stimulating device and a sensor capable of sensing photons that have been converted from a first energy to a second energy.

In some embodiments, systems disclosed herein may further comprise a means for providing to the cell-stimulating device a mechanical stimulus selected from the group consisting of ultrasound, fluid flow, and pressure puffs. In some embodiments, systems disclosed herein may further comprise a generator to provide to the cell-stimulating device an electrical stimulation. In some embodiments, systems disclosed herein may further comprise a generator to provide to the cell-stimulating device an electrical stimulation. In some embodiments, systems disclosed herein may further comprise a light source capable of stimulating the cell-stimulating device and a sensor capable of sensing photons that have been converted from a first energy to a second energy. In some embodiments, systems disclosed herein may have one and only one cell-stimulating device per well of a multi-well microplate.

Aspects of the present disclosure provide methods of preparing a tissue structure capable of growing into a cell-stimulating device disclosed herein. In some embodiments, methods of preparing a tissue structure capable of growing into a cell-stimulating device comprises: growing a tissue structure on an ultra-low adhesion microplate until it has a diameter of 100 μm to 300 pm; placing the tissue structure into a cryovial containing a freezing medium, wherein the freezing medium comprises 10% dimethyl sulfoxide (DMSO), 30% Dulbecco's Modified Eagle's Medium (DMEM), and 60% Fetal Bovine Serum (FBS); freezing the cryovial containing the tissue structure at a controlled rate of 1 degree Celsius per minute overnight in a negative 80 degree Celsius freezer; and transferring the cryovial from the negative 80 degree Celsius freezer to liquid nitrogen. In some embodiments, methods disclosed herein further comprise placing the cryovial in a 37 degrees Celsius water bath. In some other embodiments, methods disclosed herein further comprise mechanically removing ice crystals within 5 minutes of placing the cryovial in the water bath. In some embodiments, methods disclosed herein further comprise transferring the tissue structure to a culture medium comprising the mixture 98% Dulbecco's Modified Eagle's Medium (DMEM) and 2% Fetal Bovine Serum (FBS) and transferring the tissue structure and the culture medium to an ultra-low-adhesion microplate. In some embodiments, methods disclosed herein further comprise culturing the tissue structure after thawing for at least an additional 4 days.

Aspects of the present disclosure provide for methods of determining the cardiotoxicity of a drug. In certain embodiments, methods of determining the cardiotoxicity of a drug may comprise: a) assembling cells into three-dimensional spheroids from a homogeneous cell suspension, wherein the homogeneous cell suspension comprises non-excitable cells exogenously expressing one or more ion channels; b) placing the three-dimensional spheroids on top of a two-dimensional layer of cardiac tissue, wherein the cardiac tissue comprises human stem-cell-derived cardiomyocytes (hiPSC-CMs); c) allowing the three-dimensional spheroids and cardiac tissue to form spheroid-syncytia constructs; d) adding the drug to the spheroid-syncytia constructs; e) stimulating the one or more ion channels exogenously expressed by at least one external mechanism; f) measuring functional response to external stimulation in the spheroid-syncytia constructs compared to functional response to external stimulation in control spheroid-syncytia constructs, wherein control spheroid-syncytia constructs are not treated with the drug; and g) determining the cardiotoxicity of the drug by assessing the difference in functional response compared to the in functional response of the control spheroid-syncytia constructs. In some embodiments, methods disclosed herein may further comprise loading one or more magnetic nanoparticles into the three-dimensional spheroids. In some aspects, the non-excitable cells exogenously expressing one or more ion channels may comprise voltage-gated ion channels, mechanosensitive ion channels, opsins, or any combination thereof In some aspects, the voltage-gated ion channels may comprise Kir2.1, Nav1.5, or both. In some aspects, the mechanosensitive ion channels may comprise Piezol. In some aspects, the opsins may comprise ChR2. In some aspects, the one or more magnetic nanoparticles may be dextran iron-oxide composites.

In some embodiments, methods of determining the cardiotoxicity of a drug may comprise an external stimulation which can comprise an electrical actuation, optical actuation, magnetic actuation, ultrasound actuation, mechanical actuation, or any combination thereof. In some embodiments, methods of determining the cardiotoxicity of a drug may comprise a functional response to external stimulation that comprises action potential duration (APD), calcium transient duration (CTD), or any combination thereof. In some aspects, the hiPSC-CMs used in the methods disclosed herein may be derived from a subject in need of the drug being tested for cardiotoxicity.

The foregoing is intended to be illustrative and is not meant in a limiting sense. Many features and subcombinations of the present inventive concept may be made and will be readily evident upon a study of the following specification and accompanying drawings comprising a part thereof. These features and subcombinations may be employed without reference to other features and subcombinations.

### BRIEF DESCRIPTION OF THE FIGURES

The following drawings form part of the present specification and are included to further demonstrate certain embodiments of the present disclosure. Certain embodiments can be better understood by reference to one or more of these drawings in combination with the detailed description of specific embodiments presented herein.

FIGS. 1A-1B depict schematics illustrating tandem-cell-unit approach using three-dimensional spark cell spheroids. FIG. 1A illustrates the tandem-cell-unit (TCU) approach. (1) Blue light (470 nm) induces conformational change in ChR2 expressed in a non-excitable cell resulting in (2) inward current and an elevated membrane potential in the non-excitable cell. Because there exists (3) some form of coupling between the non-excitable and cardiac cells, (4) the system evokes an action potential and subsequent contraction in the cardiomyocytes. FIG. 1B shows the deployment of TCU using a spheroid structure and cardiac tissue—a schematic representation of a ChR2-expressing three-dimensional cell spheroid on top of a two-dimensional layer of hiPS-CM tissue. Confocal microscopy captured the relative positioning (ChR2-HEKs labeled green and hiPSC-CMs labeled red) as shown in the inset.

FIGS. 2A-2B depict schematics illustrating a method of spheroid formation. FIG. 2A shows microplates featuring rounded, ultra-low attachment surfaces that facilitated the formation of a three-dimensional spheroid of cells from a homogeneous cell suspension. FIG. 2B shows spheroids that were transferred in medium via standard micropipettors fitted with wide-mouthed tips.

FIGS. 3A-3I depict schematics and graphs illustrating morphological characterization of three-dimensional spheroids as function of cell density. FIG. 3A shows assembled “large” spheroid constructs initially seeded at (G1) 2×10⁴ cells/well, (G2) 4×10⁴ cells/well, (G3) 6×10⁴ cells/well, (G4) 8×10⁴ cells/well, and (G5) 10×10⁴ cells/well. Both brightfield images and fluorescence images (to confirm expression of ChR2-eYFP) were taken every 24 hours over 4 days. Scale bar is 0.5 mm. FIG. 3B shows standard imaging tools used to contour a five-point ellipse to the outline of the imaged spheroid. Axes were then used to calculate average radius and ellipticity for each spheroid over 4 days. FIG. 3C shows radius [(major axis+minor axis)/2] as a function of time. FIG. 3D shows shape (major axis/minor axis) as a function of time. For FIGS. 3C-3D, the sample size was 30 spheroids, n=6 per density and per time point. FIG. 3D shows assembled “small” spheroids with initial seeding of 10² and 10³ cells per well in a 96-well format—brightfield and eYFP fluorescence images shown at days 1 and 5. Scale bar is 0.2 mm. FIGS. 3E-3F show radius and ellipticity over 16 days. The sample size was 12 spheroids, n=6 per density and per time point. FIG. 3G shows assembled “small” spheroids with initial seeding of 5×10² and 5×10³ cells per well in a 384-well format—brightfield and eYFP fluorescence images shown at days 1 and 5. FIGS. 3H-3I show radius and ellipticity over 16 days. The sample size was 12 spheroids, n=6 per density and per time point. Scale bar is 0.2 mm for day 1 and 0.5 for day 5. In panels (B,C,E,F,H,I), all data points were overlaid in addition to the mean and standard deviation. See FIGS. 11A-11B, 12A-12B, 13A-13B, and 14A-14B as a function of time. See FIGS. 17A-17B for complete small spheroid, 96-well and 384-well format datasets. See text and FIG. 18 for details on statistics.

FIGS. 4A-4D depict images and graphs illustrating spheroid deployment and coupling to human iPSC-CM syncytia. FIG. 4A shows a representative immunofluorescent image of a spheroid deposited onto a hiPSC-CM layer. Alpha-actinin (red) identifies the actin filaments in the sarcomeres of the myocytes; Hoechst (blue) labels the cell nuclei; eYFP (green) reporter identifies the expression of ChR2 which is localized in the HEK cell membrane. Left scalebar is set to 0.5 mm. Right scalebar is at higher magnification set to 0.1 mm. FIG. 4B shows quantification of gap junction (GJA1) gene expression via qPCR (normalized by GAPDH). Shown are data from n=7 independent samples of WT HEK and ChR2-HEK cells grown in monolayers and in spheroids, as well as n=2 samples of hiPSC-CMs. FIGS. 4C-4D show Western blot and quantification (by capillary-based Wes and by standard electrophoresis-based WB) of Cx43 gap junctional protein from WT HEK and ChR2-HEK cells, normalized by GAPDH protein. In the Wes quantification, n=6 per group, plus WT HeLA cells as a negative control; in the standard WB, n=4 per group. Significant differences (p<0.05) are indicated by a (*). ****p<0.0001.

FIGS. 5A-5D depict images and graphs illustrating functional responses from human iPSC-CMs upon spark-cell cluster pacing obtained by all-optical electrophysiology. FIG. 5A shows a typical field-of-view for microscopic optical stimulation experiments featuring brightfield and corresponding fluorescence images. The middle image was produced using a mCherry filter and demonstrates expression of optogenetic sensor for calcium (R-GECO) in the iPSC-CM monolayer. The rightmost image was produced with a YFP filter and shows a portion of the ChR2-eYFP spheroid. Scale bar is 0.1 mm. FIG. 5B shows that, for recordings, the field-of-view was separated into two non-overlapping areas—patterned optical stimulation including the spheroid and optical recording including only iPSC-CMs labeled with voltage or calcium indicators. FIG. 5C shows calcium transients from R-GECO-expressing iPSC-CMs showing spontaneous activity and optical pacing (via the spheroid) at three different frequencies after 7 hours of spheroid introduction. While a 1:1 stimulation-to-response was observed for optical pacing at 0.5 and 0.75 Hz, overdrive pacing at 1.0 Hz results in failure of iPS-CMs to fully restore calcium stores between stimulation events, hence calcium alternans were seen. FIG. 5D shows optical pacing confirmed via spectrally compatible small-molecule voltage dye. This sample showed a 1:1 relationship between stimulation and response for pacing frequencies up to 1.5 Hz after 12 hours of spheroid integration.

FIGS. 6A-6B depict images illustrating macroscopic optical mapping of optically triggered spheroid-mediated calcium waves in human iPS-CMs. FIG. 6A shows calcium waves (by Rhod-4) triggered by optical light pulses at 0.5 Hz through a pair of spheroids after 24 hours integration. Activation map shows the origin of early activation (blue), red arrows indicate the locally triggered activity by the two spheroids; isochrones are 0.03 seconds (s) apart. Global calcium transients and the superimposed optical pulses are shown on the right. FIG. 6B shows calcium waves by optogenetic sensor R-GECO, triggered at 0.5 and at 1 Hz through a single spheroid (after 24 hour integration). In the activation maps (isochrones 0.03 s apart) slight wave-slowing is visible at the higher pacing frequency. At the bottom, an ROI trace is shown for the uninterrupted pacing and traces from different segments away from the spheroid illustrate sequential activation (a, b, and c). Scale bar is 1 mm.

FIGS. 7A-7B depict images illustrating a representative time course of integration of spark-cell clusters with human iPS-CM monolayers for pacing capture. FIG. 7A shows an experimental preparation timeline including separate treatment of cell lines (hiPSC-CM in blue and ChR2 293Ts in green) before acute functional studies. FIG. 7B shows that, for an early integration study, 18 samples were tested and the percentage of those samples from which we observed full pacing capture was plotted every 2 hours. An increase in the number of optical pacing responders was observed over 10 hours. The inset represents the combined results over multiple cell cultures, summarized as a percent of samples that responded to optical pacing at 6 hours (n=54) and at 24 (n=16) after spheroids were seeded onto hiPSC-CM syncytia.

FIGS. 8A-8E depict images and graphs illustrating functional responses via cryopreserved spark-cell clusters. FIG. 8A shows a schematic representation of the spheroid cryopreservation process, both freezing and thawing. FIG. 8B shows sample spheroids (1 sample seeded in a 384-well spheroid plate at 5×102 cells/well and 2 samples seeded in a 96-well spheroid plate at 10³ cells/well) from which functional data was obtained. Images (ChR2-eYFP and PI) represent spheroids 1 day before freezing and 6 days after thawing (or 1 day before functional measurements were performed). Scale bar is 0.2 mm. For each depicted spheroid are the corresponding calcium traces from jRGECO-expressing iPSC-CMs show spontaneous activity and responses to optical pacing by a spheroid at three frequencies 18 hours after spheroids were applied. Stimulation at 0.5 and 0.75 Hz resulted in a 1:1 response ratio while varying patterns alternans were observed for these samples at 1.0 Hz. FIG. 8C shows that, of the thawed spheroids that were introduced to iPSC-CMs, all (100%) of the samples demonstrated optical pacing. FIG. 8D shows a strength-duration (blue light intensity, pulse width) curve was constructed for 6/9 of the responsive cryopreserved samples at six discrete pulse widths: 40, 20, 10, 5, 1, and 0.5 ms. FIG. 8E shows for one of these six samples, a strength-duration heat map was constructed at multiple frequencies (0.5, 0.75, 1.0, and 1.5 Hz) where blue light intensity threshold (mW/mm²) for stimulation increases as pulse width decreases and/or frequency increases. Panel (A) was created using Biorender.

FIG. 9 depicts images illustrating cardiomyocyte cell spheroids assembled in U-shaped 96-well plates using human iPSC-CMs where. after day 5, an optogenetically- and magnetically-active cell spheroid was deposited onto each 35 mm dish and the dish was positioned onto a magnetic plate to pull down the spheroid for quick integration with the cardiomyocyte layer.

FIG. 10 depicts images illustrating optogenetically- and magnetically-active cell spheroids prepared using both magnetic nanoparticles (0.2 μm diameter, endocytosed) or magnetic microparticles.

FIGS. 11A-11B depict images illustrating data used in FIG. 3A, 3B, 3C from 24 hours after plating. FIG. 11A shows measurements (size and shape) for each spheroid taken 24 hours after ChR2-HEK suspension was seeded into 96-well spheroid microplates at various densities. Group 1 (G1) denotes an initial seeding density of 2×104 cells/well, Group 2 (G2) 4×104 cells/well, Group 3 (G3) denotes an initial seeding density of 6×104 cells/well, Group 4 (G4) 8×104 cells, and Group 5 (G5) denotes an initial seeding density of 10×104 cells/well. For each seeding density, n=6. FIG. 11B shows corresponding brightfield and fluorescence images at 24 hours for each spheroid from which measurement analysis in (A) was performed. Scalebar: 0.5 mm.

FIGS. 12A-12B depict images illustrating data used in FIG. 3A, 3B, 3C 48 hours after plating. FIG. 12A shows measurements (size and shape) for each spheroid taken 48 hours after ChR2-HEK suspension was seeded into 96-well spheroid microplates at various densities. Group 1 (G1) denotes an initial seeding density of 2×104 cells/well, Group 2 (G2) 4×104 cells/well, Group 3 (G3) denotes an initial seeding density of 6×104 cells/well, Group 4 (G4) 8×104 cells, and Group 5 (G5) denotes an initial seeding density of 10×104 cells/well. For each seeding density, n=6. B: FIG. 12B shows corresponding brightfield and fluorescence images at 48 hours for each spheroid from which measurement analysis in (A) was performed. Scalebar: 0.5 mm.

FIGS. 13A-13B depict images illustrating data used in FIG. 3A, 3B, 3C 72 hours after plating. FIG. 13A shows measurements (size and shape) for each spheroid taken 72 hours after ChR2-HEK suspension was seeded into 96-well spheroid microplates at various densities. Group 1 (G1) denotes an initial seeding density of 2×104 cells/well, Group 2 (G2) 4×104 cells/well, Group 3 (G3) denotes an initial seeding density of 6×104 cells/well, Group 4 (G4) 8×104 cells, and Group 5 (G5) denotes an initial seeding density of 10×104 cells/well. For each seeding density, n=6. FIG. 13B shows corresponding brightfield and fluorescence images at 72 hours for each spheroid from which measurement analysis in (A) was performed. Scalebar: 0.5 mm.

FIGS. 14A-14B depict images illustrating data used in FIG. 3A, 3B, 3C 96 hours after plating. FIG. 14A shows measurements (size and shape) for each spheroid taken 96 hours after ChR2-HEK suspension was seeded into 96-well spheroid microplates at various densities. Group 1 (G1) denotes an initial seeding density of 2×104 cells/well, Group 2 (G2) 4×104 cells/well, Group 3 (G3) denotes an initial seeding density of 6×104 cells/well, Group 4 (G4) 8×104 cells, and Group 5 (G5) denotes an initial seeding density of 10×104 cells/well. For each seeding density, n=6. FIG. 14B shows corresponding brightfield and fluorescence images at 96 hours for each spheroid from which measurement analysis in (A) was performed. Scalebar: 0.5 mm.

FIGS. 15A-15B depict images and graphs illustrating the viability of spheroids in culture over time, where spheroids seeding at a density of 2×10⁴ cells/well were cultured in a sterile propidium iodide (PI) solution of concentration 2 μg/mL in DMEM. FIG. 15A shows WT 293T and ChR2 293T spheroids cultured without PI. FIG. 15B shows WT 293T and ChR2 293T spheroids cultured with PI. Spheroids were imaged every 24 hours under brightfield and fluorescence to reveal the PI uptake localization in the necrotic core of the spheroid. In both 293T spheroids and ChR2-293T spheroids top-down images, an increase in the fraction of PI pixels to total spheroid pixels (as determined from brightfield) was observed after day 2. These fractions were quantified over time, n=3 spheroids per time point per group, all data points shown. Scalebar: 0.5 mm.

FIG. 16 depicts top-down spheroid images that were run through an intensity threshold filter in order to attain the fraction of PI-stained cells. Here PI fraction was defined as the number of PI pixels with intensity above a specified threshold in the fluorescent channel over the number of pixels constituting the spheroid as determined in brightfield. The auto-detect ROI feature available with the imaging tool package was used to selectively binarize pixels that represented the spheroid. PI fraction was subsequently calculated from the processed images of each respective channel.

FIGS. 17A-17B depict images illustrating data used in FIG. 3D, 3E, 3F, 3G, 3H, 3I (1-16 days after plating). FIG. 17A shows fluorescence (ChR2-eYFP) images at 8 time points over 16 days for spheroids seeded in 96-well ULA plates at (G6) 10² cells/well and (G7) 10³ cells/well. FIG. 17B shows fluorescence (ChR2-eYFP) at 8 time points over 16 days for spheroids seeded in 384-well ULA plates at (G6) 10² cells/well and (G7) 10³ cells/well. For each seeding density and each day, n=6. Size and shape measurements were calculated from the images via MATLAB. Scalebars are 0.2 mm at higher (10×) magnification and 0.5 mm at lower (4× magnification).

FIG. 18 illustrates tables showing all statistics for morphological data used in FIGS. 3A-3I. Two-way ANOVAs with post-hoc Tukey or Sidak (p<0.05) was conducted for evaluating radius and ellipticity as a function of initial seeding density and days in culture for large spheroids seeded in 96-well plates, small spheroids seeded in 96-well plates, and small spheroids seeded in 384-well plates. Normality of the data was also tested. Show are multiple comparisons of size and shape across seeding densities for first and last days in culture.

FIG. 19 depicts top-down spheroid images wherein the spheroids have been loaded with increasing concentrations of magnetic nanoparticles (MPs).

### DEFINITIONS

Terms, unless defined herein, have meanings as commonly understood by a person of ordinary skill in the art relevant to certain embodiments disclosed herein or as applicable.

As used herein “about” unless otherwise indicated, applies to all numbers expressing quantities of agents and/or compounds, properties such as molecular weights, reaction conditions, and as disclosed herein are contemplated as being modified in all instances by this term. Accordingly, unless indicated to the contrary, the numerical parameters in the specification and claims are approximations that can vary from about 10% to about 15% plus and/or minus depending upon the desired properties sought as disclosed herein. Numerical values as represented herein inherently contain standard deviations that necessarily result from the errors found in the numerical value's testing measurements.

As used herein, “individual”, “subject”, “host”, and “patient” can be used interchangeably herein and refer to any mammalian subject for whom diagnosis, treatment, prophylaxis or therapy is desired, for example, humans, pets, livestock, horses or other animals. In some embodiments, a subject may be a rodent, e.g., a mouse, a rat, a guinea pig, etc. In other embodiments, a subject may be a livestock animal. Non-limiting examples of suitable livestock animals may include pigs, cows, horses, goats, sheep, llamas and alpacas. In yet other embodiments, a subject may be a companion animal. Non-limiting examples of companion animals may include pets such as dogs, cats, rabbits, and birds. In yet other embodiments, a subject may be a zoological animal. As used herein, a “zoological animal” refers to an animal that may be found in a zoo. Such animals may include non-human primates, large cats, wolves, and bears. In other embodiments, the animal is a laboratory animal. Non-limiting examples of a laboratory animal may include rodents, canines, felines, and non-human primates. In some embodiments, the animal is a rodent. Non-limiting examples of rodents may include mice, rats, guinea pigs, etc. In preferred embodiments, the subject is a human.

As used herein, the terms “therapeutically effective amount” or “therapeutically effective dose,” or similar terms used herein are intended to mean an amount of an agent (e.g., chemotherapy, targeted treatments) and/or a therapy (e.g., radiation, surgery) that will elicit the desired biological or medical response (e.g., an improvement in one or more symptoms of a cancer).

The terms “treat,” “treating,” and “treatment,” as used herein, refer to therapeutic or preventative measures described herein. The methods of “treatment” employ administration to a subject, in need of such treatment, a human antibody of the present disclosure, for example, a subject in need of an enhanced immune response against a particular antigen or a subject who ultimately may acquire such a disorder, in order to prevent, cure, delay, reduce the severity of, or ameliorate one or more symptoms of the disorder or recurring disorder, or in order to prolong the survival of a subject beyond that expected in the absence of such treatment. Accordingly, “treat,” “treating,” or “treatment” can refer to reversing, ameliorating, or inhibiting onset or inhibiting progression of a health condition or disease or a symptom of the health condition or disease (e.g., cancer).

As used herein, “marker” can refer to any molecule that can be measured or detected, for example. For example, a marker can include, without limitations, a nucleic acid, such as, a transcript of a gene, a polypeptide product of a gene, a glycoprotein, a carbohydrate, a glycolipid, a lipid, a lipoprotein, a carbohydrate, and/or a small molecule. As used herein, “expression” and grammatical equivalents thereof, in the context of a marker, can refer to the production of the marker as well as the level or amount of the marker, or the cellular localization and/or accumulation of the marker within a cell.

### DETAILED DESCRIPTION

In the following sections, certain exemplary compositions and methods are described in order to detail certain embodiments of the invention. It will be obvious to one skilled in the art that practicing the certain embodiments does not require the employment of all or even some of the specific details outlined herein, but rather that concentrations, times and other specific details can be modified through routine experimentation. In some cases, well known methods, or components have not been included in the description.

The accelerating pace of drug discovery has spawned an increasing need for functional in vitro assays using living human cells. Automating these assays for high-throughput systems, however, has proven to be difficult. The present disclosure is based, at least in part, on the discovery of “spark-cell” spheroids that are usable as a “reagent” which may eventually be stored, transported and deployed on site to confer optical pacing of cardiac tissue. Such “spark-cell” spheroids are amenable to automation and can be handled robotically. When deposited onto human iPSC-CM syncytia, the spheroids provided by the present disclosure may create a spatially localized pacing site. Methods disclosed herein approach may have faster integration for acute pacing experiments compared to direct genetic modification of the cardiomyocytes and spheroids disclosed herein can be integrated in a robotics-amenable workflow for high-throughput drug testing.

The present disclosure relates generally to cell-stimulating devices or cells and cell cultures including cell-stimulating devices and target cells. The present disclosure also relates to methods of preparing cell cultures including cell-stimulating devices and target cells. Methods of analyzing cellular response(s) of target cells to electrical stimulation, optical stimulation, mechanical stimulation, and/or magnetic stimulation using the cell culture systems are also disclosed.

In some embodiments, the present disclosure provides for spheroids and compositions comprising spheroids. A “spheroid” or a “spheroid culture system” provides a similar physicochemical environment in vivo by facilitating cell—cell and cell—matrix interactions that overcome the limitations of traditional monolayer cell culture. In suspension cultures, aggregates of adjacent cells may generally form a spheroid shape, giving rise to a 3D culture system, having wide utility in, for example but not limited to, cardio-disease research, therapeutic transplantation, drug screening, and clinical study, as well as organic culture.

In certain embodiments, the present invention provides methods for preparing spheroids. In some embodiments, primary cells, stem cells, genetically modified cells, or any combination thereof can be used to prepare spheroids. In some embodiments, an immortal cell line can be used to prepare spheroids. In some embodiments, cells used to prepare spheroids according to methods disclosed herein may be endogenously non-excitable cells. As used herein, non-excitable cell types are characterized by an inability to generate all-or-none action potentials in response to depolarizing stimuli. For example, the non-excitable cell may be at least one of a HeLa cell, a CHO cell, an NIH/3T3 cell, a HEK293 cell, a fibroblast, a mesenchymal stem cell, an iPSC, an embryonic stem cell, a progenitor cell, any somatic non-excitable cell, and/or any other non-excitable cell.

Cardiac excitability arises from organized flow of ionic currents through ion-specific channels in the cell membrane, through the myoplasm and gap junctions that connect cells, and through the extracellular space. Each ion flow (current) possesses distinguishing kinetics, biochemical, or pharmacological properties, based upon the permeantion. These currents also determine the intracellular concentration of various ions and determine the potential across the cellular membrane (membrane potential).

In some embodiments, a non-excitable cell that does not endogenously express one or more proteins and/or genes that convey the ability of the cell to respond to an external electrical, optical, magnetic, and/or ultrasound/mechanical actuation may be used to prepare spheroids according to the methods herein. In some aspects, these cells may be genetically modified to express one or more proteins and/or genes that convey the ability to respond to an external electrical, optical, magnetic, and/or ultrasound/mechanical actuation.

In some embodiments, cells for use in preparing spheroids according to the methods herein may have been genetically modified to express or over express one or more Ca⁺² channels, Na⁺ channels, K⁺ channels, or any combination thereof. These channels/transporters affect the action potential of the cardiac cells by altering Ica, INa, or IK. Accordingly, cells disclosed herein genetically modified to express or overexpress one or more Ca⁺² channels, Na⁺ channels, K⁺ channels, or any combination thereof may respond to external electrical actuation. Non-limiting examples of cardiac ion channels can include Nav1.5, Nav1.8, Cav1.2, Cav1.3, Cav2.2, Kv4.3, Kv1.4, Kv1.5, Kv11.1, Kv7.1, Kir2.1, Kir3, Kir6, or any combination thereof In some embodiments, cells for use in preparing spheroids according to the methods herein may have been genetically modified to express or overexpress Kir2.1, Nav1.5, or both.

In some embodiments, cells for use in preparing spheroids according to the methods herein may have been genetically modified to express or over express one or more opsins. Opsins are light-gated ion channels or pumps that absorb light at specific wavelengths. Upon activation by light, these channels and pumps respond by opening or closing, which conducts the flow of ions into or out of the cell. Accordingly, cells disclosed herein genetically modified to express or overexpress one or more opsins may respond to external optical actuation. Non-limiting examples of opsins can include channelrhodopsins (e.g., Channelrhodopsin-1 (ChR1), Channelrhodopsin-2 (ChR2), ChR2(H134R), C1V1(t/t), ChIEF, ChETA, C1V1(t/t), ChrimsonR, VChRl, C1V1(t/t), Chrimson, ChrimsonR, Chronos), halorhodopsins (e.g., Jaws, Halo/NpHR, eNpHR 3.0), archaerhodopsins (e.g., Arch, Arch-3), Leptosphaeria rhodopsin (e.g., Mac), and the like. In some embodiments, cells for use in preparing spheroids according to the methods herein may have been genetically modified to express or overexpress one or more channelrhodopsins. In some aspects, cells for use in preparing spheroids according to the methods herein may have been genetically modified to express or overexpress Channelrhodopsin-2 (ChR2).

In some embodiments, cells for use in preparing spheroids according to the methods herein may have been genetically modified to express or over express one or more mechanosensitive ion channels. A mechanosensitive ion channel is a transmembrane protein, which uses external mechanical force to bias its open probability and therefore the amount of ions it lets through. Accordingly, cells disclosed herein genetically modified to express or overexpress one or more mechanosensitive ion channels may respond to external mechanical actuation. Non-limiting examples of mechanosensitive ion channels can include TRP channels, TRPV4, TRPC1, TRPC3, TRPC6, Piezol, and the like. In some embodiments, cells for use in preparing spheroids according to the methods herein may have been genetically modified to express or overexpress Piezol.

One of skill in the art will appreciate that any method of genetically modifying a cell as disclosed herein to express or overexpress one or more proteins and/or genes (e.g., Ca⁺² channels, Na⁺ channels, K⁺ channels, opsins, mechanosensitive ion channels) can be used. In some embodiments, nucleic acids encoding the one or more channels may be delivered to a non-excitable cell by viral infection using, for example, adenovirus, adeno-associated virus (AAV), and lentivirus; electroporation; liposome-mediated transfection (lipofection); transfection using a chemical transfection reagent; heat shock transfection; microinjection, and the like. Cells may be subsequently subjected to any assay suitable for determining changes in protein expression levels using methods such as, but not limited to, Western blotting, ELISA, IHC, and the like; or for determining changes in gene expression levels using methods such as, but not limited to, RT-PCR, qRT-PCT, Northern blot, and the like.

In certain embodiments, spheroids can be prepared using any of the cells disclosed herein. In some embodiments, methods of preparing spheroids disclosed herein may yield spheroids having a diameter size of about 20 mm or less, about 15 mm or less, about 10 mm or less, about 5 mm or less, about 2.5 mm or less, about 1 mm or less, or about 0.5 mm or less. In some embodiments, spheroids disclosed herein may have a diameter size of about 20 mm, about 15 mm, about 10 mm, about 5 mm, about 10 mm, about 5 mm, about 2.5 mm, about 1 mm, about 0.9 mm, about 0.8 mm, about 0.7 mm, about 0.6 mm, about 0.5 mm, about 0.4 mm, about 0.3 mm, about 0.2 mm, or about 0.1 mm. In some embodiments, methods of preparing spheroids disclosed herein may yield a composition comprising about 100% to about 10% of spheroids having the same diameter size. In some embodiments, methods of preparing spheroids disclosed herein may yield a composition comprising at heterogenous mixture of spheroids having diameter sizes ranging from of about 20 mm to about 0.1 mm, about 15 mm to about 0.2 mm, or about 10 mm to about 0.3 mm.

As used herein, the term “seed” refers to the addition of desired cells to a medium prepared for the culturing of cells. For preparing spheroids at efficient sizes according to the method disclosed herein, the seeding density may be between about 1×10² to about 2×10⁷ cells/well, wherein a well is a well in a 96 well plate which has a surface area of 0.32 cm². In some embodiments, the seeding density may be about 1×10² cells/well, about 1×10³ cells/well, about 1×10⁴ cells/well, about 1×10⁵ cells/well, about 1×10⁶ cells/well, about 1×10⁷ cells/well, about 1×10⁸ cells/well, about 1×10⁹ cells/well, about 2×10² cells/well, about 2×10³ cells/well, about 2×10⁴ cells/well, about 2×10⁵ cells/well, about 2×10⁶ cells/well, or about 2×10⁷ cells/well, wherein a well is a well in a 96 well plate which has a surface area of 0.32 cm².

In some embodiments, methods of preparing spheroids disclosed herein may reach the desired spherical shape and or size after about 1 day to about 4 weeks in culture. In some embodiments, methods of preparing spheroids disclosed herein may reach the desired spherical shape and or size after about 1 day, after about 2 days, after about 3 days, after about 4 days, after about 5 days, after about 6 days, after about 7 days, after about 8 days, after about 9 days, after about 10 days, after about 11 days, after about 12 days, after about 13 days, after about 14 days, after about 3 weeks, or after about 4 weeks in culture.

In some embodiments, spheroids prepared according to the methods herein may be loaded with magnetic particles. Spheroids loaded with magnetic particles can allow for sequential aggregation of magnetically labelled spheroids followed by actuation by a magnetic field which produces localized magnetic clusters within the spheroids. These clusters impose local mechanical forces on the surrounding tissue in response to applied global magnetic fields. Herein, use of “magnetic actuation” is a positioning force, unlike the electrical, optical, and mechanical actuation, which all involve some ion transport event that then alters the electrical potential of the entity to which the spheroid is coupled. In some embodiments, magnetic particles for loading into spheroids prepared according to the methods herein may be magnetic nanoparticles, magnetic microparticles, or any combination thereof In some aspects, magnetic nanoparticles for use herein may range from about 1 nm to about 500 nm, 2 nm to about 250 nm, or about 3 nm to about 1 nm to about 200 nm in diameter size. In some other aspects, magnetic nanoparticles for use herein may be about 1 nm, about 10 nm, about 20 nm, about 30 nm, about 40 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, about 200 nm, about 210 nm, about 220 nm, about 230 nm, about 240 nm, about 250 nm, about 260 nm, about 270 nm, about 280 nm, about 290 nm, about 300 nm, about 310 nm, about 320 nm, about 330 nm, about 340 nm, about 350 nm, about 360 nm, about 370 nm, about 380 nm, about 390 nm, about 400 nm, about 410 nm, about 420 nm, about 430 nm, about 440 nm, about 450 nm, about 460 nm, about 470 nm, about 480 nm, about 490 nm, or about 500 nm in diameter size. In some embodiments, magnetic particles for use herein may be magnetic microparticles. In some aspects, magnetic microparticles for use herein may have a diameter of up to about 10 μm, up to about 50 μm, up to about 100 μm, up to about 250 μm, or up to about 500 μm. In some other aspects, magnetic microparticles for use herein may have a diameter of about 0.5 μm, about 1μm, about 2.5 μm, about 5μm, about 7.5 μm, about 10 μm, about 15 μm, about 20 μm, about 30 μm, about 40 μm, about 50 μm, about 60 μm, about 70 μm, about 80 μm, about 90 μm, about 100 μm, about 125 μm, about 150 μm, about 175 μm, about 200 μm, about 250 μm, about 300 μm, about 350 μm, about 400 μm, about 450 μm, or about 500 μm. Non-limiting examples of magnetic nanoparticles and/or magnetic microparticles for use herein can include iron oxide magnetic nanoparticles and/or magnetic microparticles, maghemite magnetic nanoparticles and/or magnetic microparticles, magnetite magnetic nanoparticles and/or magnetic microparticles, metallic magnetic nanoparticles and/or magnetic microparticles, and the like. In some aspects, magnetic nanoparticles and/or magnetic microparticles may have a shell. In some aspects, magnetic nanoparticles and/or magnetic microparticles may be crosslinked with one or more agents, compounds, tags, peptides, antibodies, oligos, and the like. In some embodiments, magnetic nanoparticles and/or magnetic microparticles for use herein may have iron oxide composites and, optionally, may be cross-linked with dextran.

In certain embodiments, spheroids loaded with magnetic particles disclosed herein may be added to one or more non-modified cardiac cells and/or non-modified cardiac tissues. In some embodiments, spheroids loaded with magnetic particles may be in contact with one or more non-modified cardiac cells and/or non-modified cardiac tissues wherein the non-modified cardiac cells and/or non-modified cardiac tissues are attached to a solid surface, and the spheroids loaded with magnetic particles may be in contact with the side of the non-modified cardiac cells and/or non-modified cardiac tissues that are not attached to the solid surface. In some embodiments, a magnet may be located underneath the solid surface that has non-modified cardiac cells and/or non-modified cardiac tissues attached to it, wherein the magnet may lock the spheroids loaded with magnetic particles in contact with the side of the non-modified cardiac cells and/or non-modified cardiac tissues that are not attached to the solid surface. In some aspects, the magnet located underneath the solid surface that has non-modified cardiac cells and/or non-modified cardiac tissues attached to it may be used to lock the spheroids loaded with magnetic particles in contact with the side of the non-modified cardiac cells and/or non-modified cardiac tissues that are not attached to the solid surface for at least about 10 minutes, at least about 20 minutes, at least about 30 minutes, at least about 40 minutes, at least about 50 minutes, or at least about 60 minutes. In some aspects, the magnet located underneath the solid surface that has non-modified cardiac cells and/or non-modified cardiac tissues attached to it may be used to lock the spheroids loaded with magnetic particles in contact with the side of the non-modified cardiac cells and/or non-modified cardiac tissues that are not attached to the solid surface for at least about 30 minutes which may allow for pacing ability in the cells within at least about 10 minutes to at least about 5 hours, at least about 30 minutes to at least about 2.5 hours, or at least about 1 hour.

In certain embodiments, spheroids loaded with magnetic particles disclosed herein can be guided along an arbitrary trajectory to a desired location using magnetic guidance. In some embodiments, a magnet may be located underneath the solid surface that can have non-modified cardiac cells and/or non-modified cardiac tissues attached to it, wherein the magnet location may be controlled by a magnetic guidance system subject to automated control. In some aspects, a magnet located underneath the solid surface may be controlled by G-code to generate one or more desired trajectories to guide one or more spheroids disclosed herein, wherein the spheroids are positioned above the magnetic guidance system. As used herein, G-code is an industry-standard language used for CNC (computer numerical control)—the automated control of machining. G-code tells the motors in CNC machines (e.g., a magnetic guidance system disclosed herein) where to move, how fast to move, and/or what path to follow. In some embodiments, a magnetic guidance system disclosed herein may guide one or more spheroids disclosed herein to a desired location and/or deploy the one or more spheroids on site for various actuation modalities. In some aspects, a magnetic guidance system disclosed herein may guide one or more spheroids that have been genetically modified to express or over express one or more Ca⁺² channels, Na⁺ channels, K⁺ channels or any combination thereof and loaded with magnetic particles as disclosed herein, to a desired location and subject the one or more spheroids to electrical actuation at the desired location. In some other aspects, a magnetic guidance system disclosed herein may guide one or more spheroids that have been genetically modified to express or over express one or opsins and loaded with magnetic particles as disclosed herein, to a desired location and subject the one or more spheroids to optical actuation at the desired location. In still some other aspects, a magnetic guidance system disclosed herein may guide one or more spheroids that have been genetically modified to express or over express one or mechanosensitive ion channels and loaded with magnetic particles as disclosed herein, to a desired location and subject the one or more spheroids to mechanical actuation at the desired location.

In certain embodiments, spheroids prepared according to the methods herein may be cryopreserved and used after subsequent thawing. In some aspects, spheroids disclosed herein may be frozen in a freezing medium. In some aspects, a freezing medium suitable for use herein may consist of about 30% DMEM, about 60% FBS, about 10% DMSO, or any combination thereof. In some embodiments, spheroids disclosed herein may be frozen and stored at about ˜80° C. to about ˜120° C. In some aspects, spheroids disclosed herein may be frozen and stored at about ˜80° C. to about ˜120° C. for up to about 1 day, up to about 4 days, up to about 1 week, up to about 2 weeks, up to about 3 weeks, up to about 4 weeks, up to about 6 weeks, up to about 2 months, up to about 4 months, up to about 6 months, up to about 8 months, up to about 10 months, up to about 1 year, or beyond without loss of functional activity and/or viability upon thawing. In some aspects, spheroids disclosed herein may be frozen and stored at about ˜80° C. to about ˜120° C. for up to about 1 day, up to about 4 days, up to about 1 week, up to about 2 weeks, up to about 3 weeks, up to about 4 weeks, up to about 6 weeks, up to about 2 months, up to about 4 months, up to about 6 months, up to about 8 months, up to about 10 months, up to about 1 year, or beyond with a loss of less than about 20% to less than about 1% of functional activity and/or viability upon thawing.

Embodiments of the present disclosure also contemplate a composite tissue comprising one or more spheroids as disclosed herein. In some embodiments, a composite tissue disclosed herein may have: 1) one or more spheroids that have been genetically modified to express or over express one or more Ca⁺² channels, Na⁺ channels, K⁺ channels, or any combination thereof; 2) one or more spheroids that have been genetically modified to express or over express one or more opsins; 3) one or more spheroids that have been genetically modified to express or over express one or more mechanosensitive ion channels; 4) one or more spheroids prepared from an un-modified cardiomyocyte (or other excitable cell); or any combination thereof In some aspects, a composite tissue disclosed herein may have: 1) one or more spheroids that have been genetically modified to express or over express one or more Ca⁺² channels, Na⁺ channels, K⁺channels, or any combination thereof; 2) one or more spheroids that have been genetically modified to express or over express one or more opsins; and/or 3) one or more spheroids that have been genetically modified to express or over express one or more mechanosensitive ion channels; and one or more spheroids prepared from an un-modified cardiomyocyte (or other excitable cell). In some other aspects, a composite tissue disclosed herein may have one or more spheroids that have been genetically modified to express or over express one or more opsins and one or more spheroids prepared from an un-modified cardiomyocyte (or other excitable cell). In still some other aspects, a composite tissue disclosed herein may have one or more spheroids that have been genetically modified to express or over express channelrhodopsin-2 (ChR2) and one or more spheroids prepared from an un-modified cardiomyocyte (or other excitable cell). In some embodiments, a composite tissue disclosed herein may have: 1) one or more spheroids that have been genetically modified to express or over express one or more Ca⁺² channels, Na⁺ channels, K⁺channels, or any combination thereof; 2) one or more spheroids that have been genetically modified to express or over express one or more opsins; 3) one or more spheroids that have been genetically modified to express or over express one or more mechanosensitive ion channels; 4) one or more spheroids prepared from an un-modified cardiomyocyte (or other excitable cell); or any combination thereof wherein any of the spheroids may be loaded with magnetic particles according to the methods disclosed herein.

Spheroids prepared according to the methods herein may be used in, but are not limited to use in, systems to assess cardiac function and/or high throughput screening systems to assess for drug cardiotoxicity. In some embodiments, spheroids disclosed herein can be used in conjunction with a variety of non-modified experimental cardiac systems. Eliminating the need for genetic transformation of the target cells and the associated efforts for optimization of gene delivery in each studied cell type constitute advantages of such systems. In some embodiments, spheroids disclosed herein can be used in “high-throughput” screenings. “High-throughput” or “HT” in the industrial setting of drug discovery and testing implies capability of performing over 10,000 assays a day. HT can require that the samples and the process are scalable, manufacturing-friendly, and amenable to handling with standard liquid and cell dispensing robotics within a standardized plate-format setting. With robotic dispensing of cells and drugs, the 96-well format, demonstrated here, can be instantly upgraded to 384-well or other standard plate formats, with simple reprogramming. In some embodiments, spheroids disclosed herein can be grown in uniquely designed plates with ultra-low attachment surfaces facilitate the formation of a three-dimensional spheroid of cells from a homogeneous cell suspension. Thus, the present system is scalable. The current implementation has built-in parallelism within a well, interrogating hundreds of cells simultaneously but generally relies on serial traversing of the wells.

Cell-stimulating devices disclosed herein may comprise a tissue structure. In some embodiments, a tissue structure encompasses one or more spheroids prepared according to the methods herein. In some embodiments, tissue structure for use in the compositions and methods herein may have a diameter of between about 0.05 mm and about 2 mm, about 0.1 mm and about 1.5 mm, or about 0.15 mm and about 1 mm. In some embodiments, a tissue structure for use in the compositions and methods herein may have a plurality of cell bodies selected from at least one of biological cell bodies or non-biological cell bodies. Cell-stimulating devices or cells may include a synthetic cell body (e.g., a liposome) or real cell body (e.g., a HeLa cell, a CHO cell, an NIH/3T3 cell, a HEK293 cell, a fibroblast, a mesenchymal stem cell, an iPSC, an embryonic stem cell, a progenitor cell, etc.). In some embodiments, a tissue structure for use in the compositions and methods herein may have a plurality of cell bodies ranging from about 10 to about 100,000 cell bodies, about 50 to about 50,000 cell bodies, or about 100 to about 10,000 cell bodies.

In some embodiments, cell bodies herein may include photon-sensitive or photon-generating entities (e.g., chromophores, nanoparticles, etc.) that have at least one of the following properties: 1) create a local electric field change induced by at least one of the following: a) a chromophore absorbing a photon and inducing a conformational change in an ion channel or other membrane-bound protein, such as ChR2 or other opsins and/or b) a nanoparticle or chromophore that is able to absorb photons and generate electric fields, such as plasmonic nanoparticles; 2) convert photons of a specific energy to a different energy by at least one of the following: a) multi-photon absorption; b) sequential-photon absorption up-conversion, e.g., up-conversion nanoparticles (UCNP); and/or c) possessing large non-linear coefficients performing second and higher-order harmonic generation, multi-wave mixing, or spontaneous parametric down-conversion; 3) emit photons without the presence of external photons, but rather the presence of a substrate, i.e., bioluminescence enzyme or hybrid (luminopsin).

Cell-stimulating devices disclosed herein producing local electric field changes can be used to alter the membrane potential of neighboring target cells without the specific need of gap-junctions between the device and target cell and/or without relying on sufficient electric field strength at the membrane of the target cell allowed by at least one of the following: a) proximity of the bio-photonic devices and target cells and/or b) intrinsic properties of the chromophores/nanoparticles.

Cell-stimulating devices disclosed herein containing energy-converting entities can be used for: activating photon-sensitive entities to induce a local electric field change, where the entities are either inside the cell-stimulating device or target cell; allowing for activation of photon-sensitive entities that are either inside the bio-photonic device or target cell at different wavelengths compared to what is intrinsically required to activate the photon-sensitive entities; generating a change in action potential or duration thereof following electrical stimulation; and/or allowing for activation of mechanosensitive ion channels in response to ultrasound, fluid flow, pressure puffs, and/or other mechanical forces.

Combined cell-stimulating device/target cell cultures or samples can be generated by sequential-in-time deposition or “sprinkling” cell-stimulating devices (i.e., spheroids) on to already cultured target cells. Sequential-in-time deposition of target cells on top of “substrates” of bio-photonic devices; and because gap junctions are not a prerequisite, the samples can be ready in less than 24 hours of combining the two cell types.

Cell-stimulating devices capable of altering membrane potentials to impart light sensitivity on target cell samples: when combined with fluorescent reporters, all-optical EP measurements can be performed; provides a means of having high spatio-temporal control of target cells; allows standardization and consistency (bio-photonic devices can be manufactured in a highly controlled manner and provided as a reagent); does not require genetic modification of the target cells and permits their direct use/study without any added complexity.

An automated system for measuring a local electric field change can be constructed that employs the cell-stimulating device/target cell samples. The system can be low-energy and miniaturized. Deposition of cell-stimulating devices (i.e., spheroids) can be fully automated using robotics. Cell-stimulating device/target cell samples combined with the automated system can be used to perform HT EP testing. Such testing can be for: ion-channel targeting drug screening; cardiotoxicity screening; cell phenotyping (e.g., functional characterization of stem-cell-derived CMs). Automated software can be used for processing the EP data obtained using the HT EP data.

An aspect of the disclosure relates to a cell-stimulating device including a photonic device or photonic entity comprising a chromophore, a nanoparticle, or a combination of both a chromophore and a nanoparticle that absorbs radiation in the near UV (100 nm-400 nm), visible (400 nm-700 nm), and/or near infrared (700 nm-1,300 nm). When exposed to light, the bio-photonic device may undergo at least one of the following processes to induce a local electric field change: i) a conformational change that results in the state-change of an ion channel present in the cell causing an influx or efflux of ions; ii) a conformational change that results in the state-change of a non-ion channel protein present in the cell; or iii) forms a dipole to generate a localized plasmonic electric field strong enough to affect neighboring cells.

Another aspect of the disclosure relates to a cell culture including the combination of one or more cell-stimulating devices (i.e., spheroids) with target cells or target excitable cells, whereby the combination of cell-stimulating devices imparts a sensitivity to external stimuli on the target cells. In some embodiments, the cell culture can include target cells that were cultured first and wherein the cell-stimulating devices were added later, and only less than about 3, about 6, about 12, or about 24 hours prior to analysis by external stimulation of the cell-stimulating devices. In certain embodiments, the cell-stimulating device may be plated first and the target cells may be added later and only less than about 3, about 6, about 12, or about 24 hours prior to analysis by external stimulation of the cell-stimulating device. The cell culture may further include a fluorescent probe or reporter that emits light in response to electrical excitation of the target cells. In some embodiments, the light emitted by the fluorescent reporter may be of a different wavelength than the wavelength of light to which a cell-stimulating device is most responsive.

Another aspect of the disclosure relates to methods for using a cell-stimulating device to impart external sensitivity at specific energies on target cells. The methods may include providing strong enough optically-induced, mechanically-induced, magnetically-induced, electrically induced, or any combination thereof electric field changes by: i) proximity between the cell-stimulating device and a target cell; ii) an adequate number of photonic entities contained in the cell-stimulating device to drive field changes; and/or iii) entities that can result in changes to the local electric field with sufficient efficiency. In certain embodiments, methods disclosed herein may use a cell-stimulating device for at least 30 minutes to at least 5 hours, for at least 45 minutes to at least 4 hours, or for at least 1 hours to at least 3 hours to impart external sensitivity at specific energies on target cells. In some embodiments, methods disclosed herein may use a cell-stimulating device for about 15 minutes, about 30 minutes, about 45 minutes, about 1 hour, about 1.5 hours, about 2 hours, about 2.5 hours, about 3 hours, about 3.5 hours, about 4 hours, about 4.5 hours, or about 5 hours to impart external sensitivity at specific energies on target cells. In some embodiments, a cell-stimulating device herein may be used to induce pacing activity in a cardiomyocyte and/or cardiac tissue. In some aspects, methods disclosed herein may use a cell-stimulating device for at least 30 minutes to at least 5 hours, for at least 45 minutes to at least 4 hours, or for at least 1 hours to at least 3 hours to induce pacing activity in a cardiomyocyte and/or cardiac tissue. In some other aspects, methods disclosed herein may induce pacing activity in a cardiomyocyte and/or cardiac tissue within about 15 minutes, about 30 minutes, about 45 minutes, about 1 hour, about 1.5 hours, or about 2 hours when spheroids loaded with magnetic particles disclosed herein are in contact with the cardiomyocyte and/or cardiac tissue, wherein the cardiomyocyte and/or cardiac tissue is attached to a solid surface and a magnet is located underneath the solid surface.

Another aspect of the disclosure relates to methods for placing a cell-stimulating device in sufficient proximity to a target cell. For example, the cell-stimulating device (i.e., spheroids) can be placed in sufficient proximity to a target cell by: a) sequential-in-time deposition or “sprinkling” of the device on to already cultured target cells; b) sequential-in-time deposition of target cells on top of “substrates” of the devices; and/or c) because gap junctions are not a prerequisite, the samples can be ready in less than 24 hours of combining the two cell types.

Another aspect of the disclosure relates to methods for performing spatial and temporal manipulation using a cell-stimulating device combined with a sample containing cells capable of firing APs (excitable cells). The methods may include, a stimulus provided by an external source to: i) activate the entities (e.g., overexpressed ion channels, magnetic nanoparticles) in a cell-stimulating device capable of altering the membrane potential of neighboring cells and/or ii) activate entities in the cell-stimulating device capable of performing an energy conversion.

Another aspect of the disclosure relates to methods for performing EP measurements in a sample. The methods may include: a) use of external stimulation and/or b) use of fluorescent reporters for reading out EP signals. Another aspect of the disclosure relates to portable, stand-alone imaging devices for performing EP measurements on a multi-well sample. The devices may include a compact imaging device including an imaging sensor, for example, that is a pixel array. The pixel array may include a CMOS detector, an EMCCD, a CCD, a single photon counting device array, an avalanche photodiode array, and/or a photomultiplier tube array. Furthermore, the devices may include adequate specifications determined by the fluorescent reporter, including: 1) a pixel well-depth capacity of sufficient size to capture the full range of the recorded signal; 2) dark counts well below the dynamic range of the recorded signal; and/or 3) a quantum efficiency large enough to capture the signal. The devices, when combined with the imaging optics, may: 1) capture activity across at least a 400 μm FOV or larger and/or 2) provide a resolution of at least 0.8 μm/pixel or smaller. The devices may include a readout rate sufficiently faster than the signal speed, a frame rate greater than about 200 frames/second, and/or may be a component of a smartphone camera.

In some embodiments, the devices may include light sources for exciting fluorescent reporters for imaging. The light sources may be incoherent sources (e.g., LEDs, including low-powered LEDs; OLED displays; and/or halogen, arc, or other high-powered incoherent sources). The light sources may be coherent sources (e.g., lasers). The light sources may be spectra having small bandwidths centered at the excitation wavelengths of the fluorescent reporter.

Another aspect of the disclosure relates to a robotic system for performing EP measurements (e.g., a fully robotic system). The system may be capable of automated: deposition of cell-stimulating devices, maintenance of samples, introduction of fluorescent reporters, and/or treatment with external compounds. The system may be controlled remotely. The system may employ use of commercially available robotic deposition systems. The system may be contained within an external housing unit.

The present disclosure provides kits for performing any of the methods disclosed herein. In some aspects, the present disclosure provides a kit for preparing any of the cell-stimulating devices (i.e., spheroids) disclosed herein. In some other aspects, the present disclosure provides a kit for determining the cardiotoxicity of a drug.

In certain embodiments, kits disclosed herein may be used for carrying out the assays described above. In some embodiments, kits disclosed herein may contain at least one ancillary agent used for carrying out the assays herein such as buffering agents, antigen retrieval solutions and reagents, and protein stabilizing agents (e.g., polysaccharides). The diagnostic kit can further comprise, where necessary, other components of the signal-producing system including agents for reducing background interference, control reagents or an apparatus or container for conducting the test.

Kits typically include a label indicating the intended use of the contents of the kit and instructions for use. The term label includes any writing, or recorded material supplied on or with the kit, or which otherwise accompanies the kit. Accordingly, this disclosure provides for kits for identifying drug cardiotoxicity prior to or during the drug's use in a clinical trial. Also provided herein are kits toward harvesting a sample from a subject, where in the sample is used to generate cardiac stem cells for use in determining an individual subject's sensitivity to a particular drug. Also provided in kits herein are regents and/or buffers need to conduct methods disclosed herein. Kits may further include guidance on suitable treatment regimens for drugs identified as having one or more cardiotoxicities and/or identifying a subject as having a cardiac-based disease, a cardiac-based disorder, and/or a cardiac-based drug sensitivity.

### EXAMPLES

The following examples are included to illustrate certain embodiments. It should be appreciated by those of skill in the art that the techniques disclosed in the examples which follow represent techniques discovered to function well in the practice of the claimed methods, compositions and apparatus. However, those of skill in the art should, in light of the present disclosure, appreciate that changes can be made in some embodiments which are disclosed and still obtain a like or similar result without departing from the spirit and scope of the invention.

### Introduction to Examples 1-8

Traditionally, cardiac tissue is stimulated using electrodes delivering electrical pulses. Such electrodes require physical contact and cannot easily be deployed for multisite stimulation. Optogenetic modification via infection or transfection to introduce light-sensitive ion channels or opsins, such as channelrhodopsin-2 (ChR2), in cardiomyocytes can allow pacing by light—offering certain benefits over the electrical stimulation currently used in the field. As such, exemplary methods herein are generally toward engineering three-dimensional “spark cells” spheroids, composed of ChR2-expressing human embryonic kidney cells and characterizing their morphology as function of cell density and time.

### Example 1

Exemplary methods as used in the illustrative examples herein are described as follows. These exemplary methods can also be used with any of the embodiments disclosed herein.

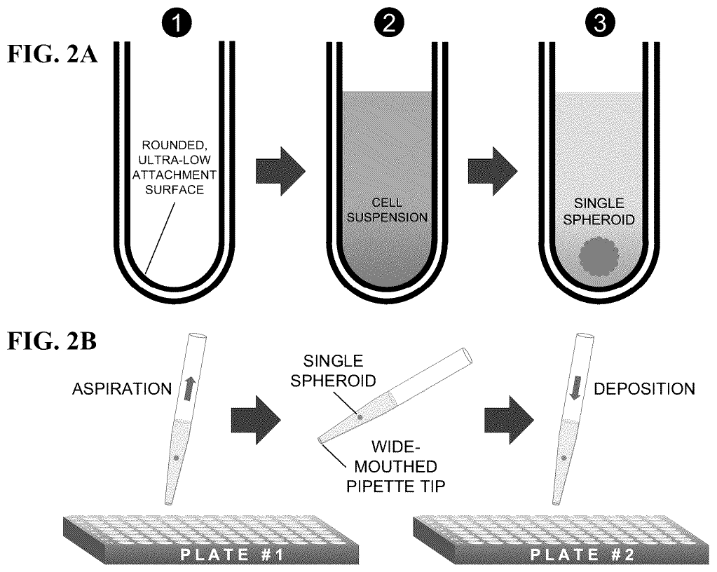

ChR2-HEK Spheroid Assembly. An immortal 293T-HEK cell line expressing ChR2 with a YFP tag devloped using Addgene construct pcDNA3.1/hChR2(H134R)-EYFP, was thawed, plated, and expanded in standard T-75 flasks in a Dulbecco's Modified Eagle Medium (DMEM) supplied with 10% fetal bovine serum (FBS) and 1% penicillin-streptomycin. ChR2-HEKs were then dissociated from the T-75 tissue culture flasks by 0.05% trypsin in Hanks' Balanced Salt Solution (HBSS) following a phosphate-buffered solution (PBS) rinse. After the resulting suspension underwent centrifugation, cells were resuspended in a volume of DMEM appropriate to yield spheroids of desired seeding density per well of a 96-well microplate (e.g., 10² to 10⁵ cells/200 μL) or 384-well microplate (e.g., 5×10² to 5×10³ cells/80 μL). Spheroids were cultured in Corning spheroid microplates in both 96-well (Catalog #CLS4920, Millipore Sigma) and 384-well (Catalog #CLS3830, Millipore Sigma) formats. These plates are uniquely designed with rounded, ultra-low-attachment surfaces to prevent cell adhesion while promoting self-assembly of cells into three-dimensional spheroids, as shown in FIG. 2A. Cell culture medium (DMEM+10% FBS+1% penicillin-streptomycin) was replaced every other day using a 50/50 approach (replace 50% of the medium) to minimize spheroid disturbance. Over 600 spheroids of different sizes were grown in different experimental rounds to optimize conditions and ultimately to produce data reported here. Growth of the spheroids was observed longitudinally via microscopy for as long as they remained viable.

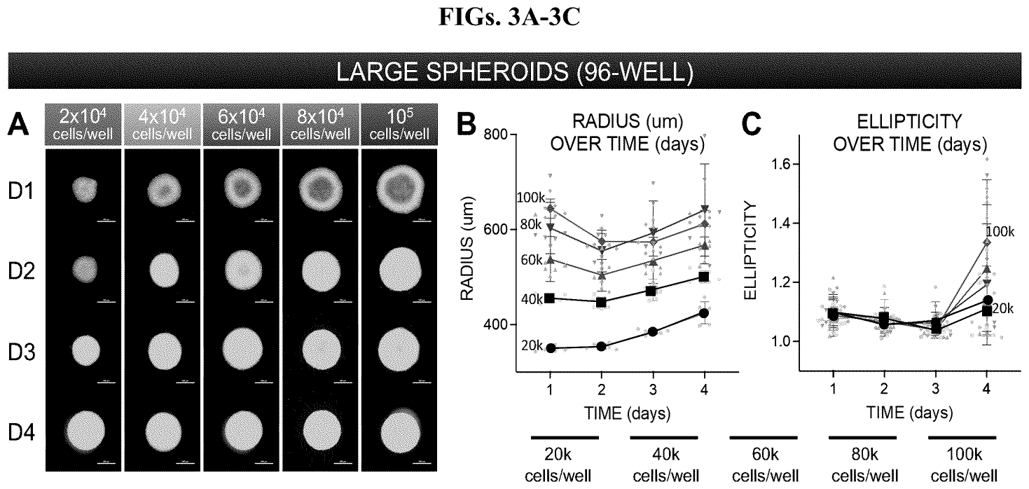

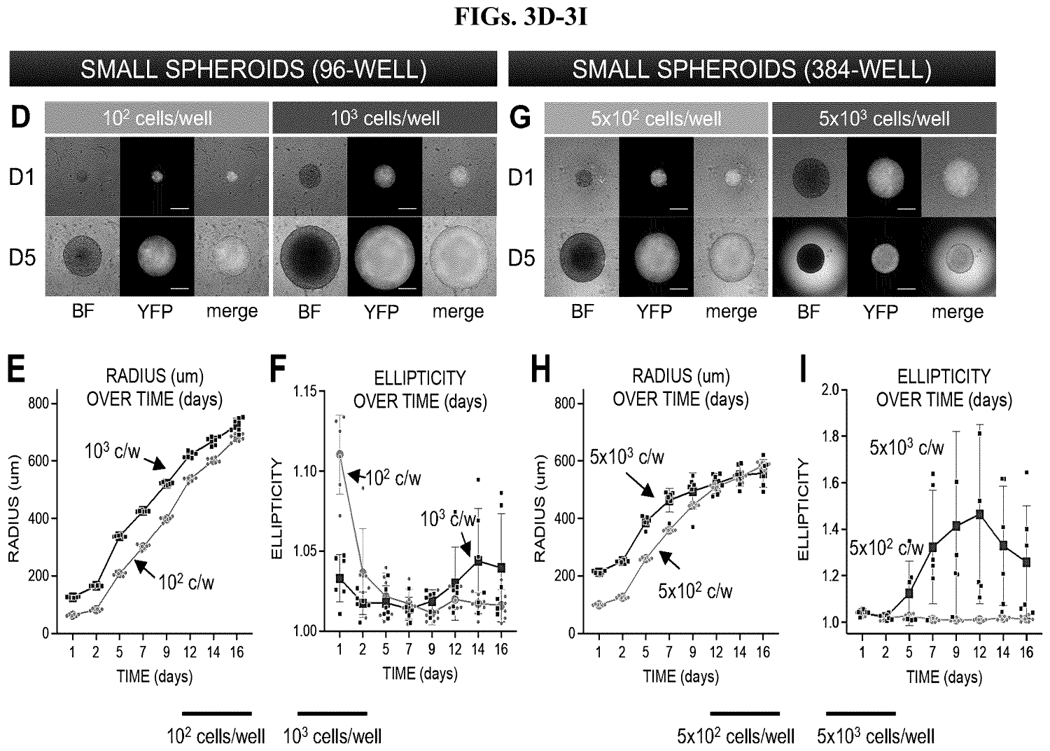

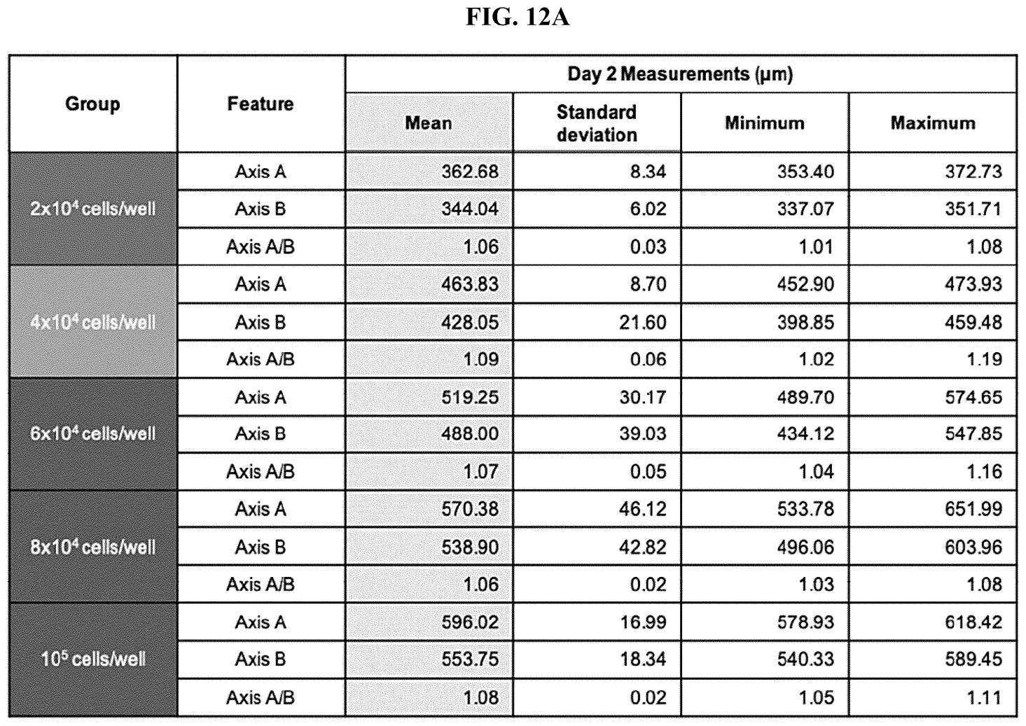

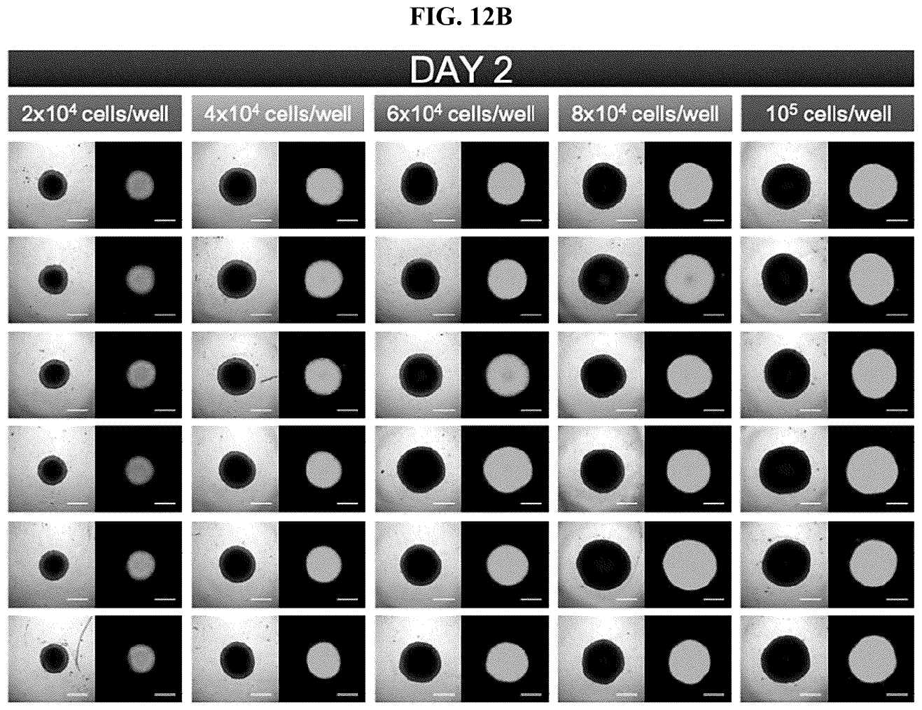

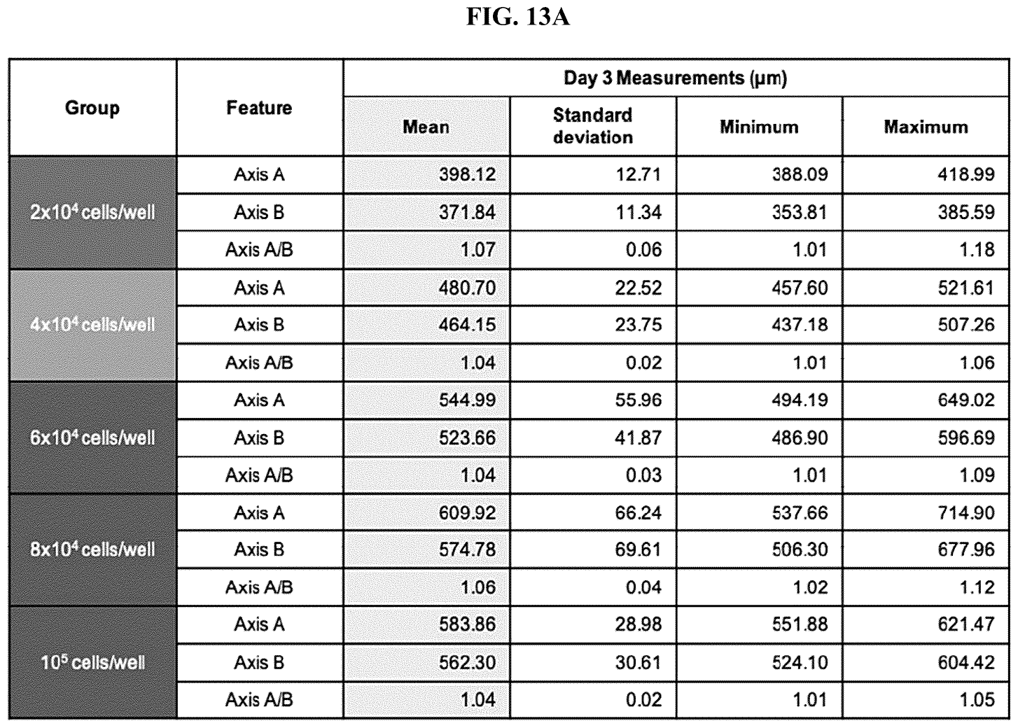

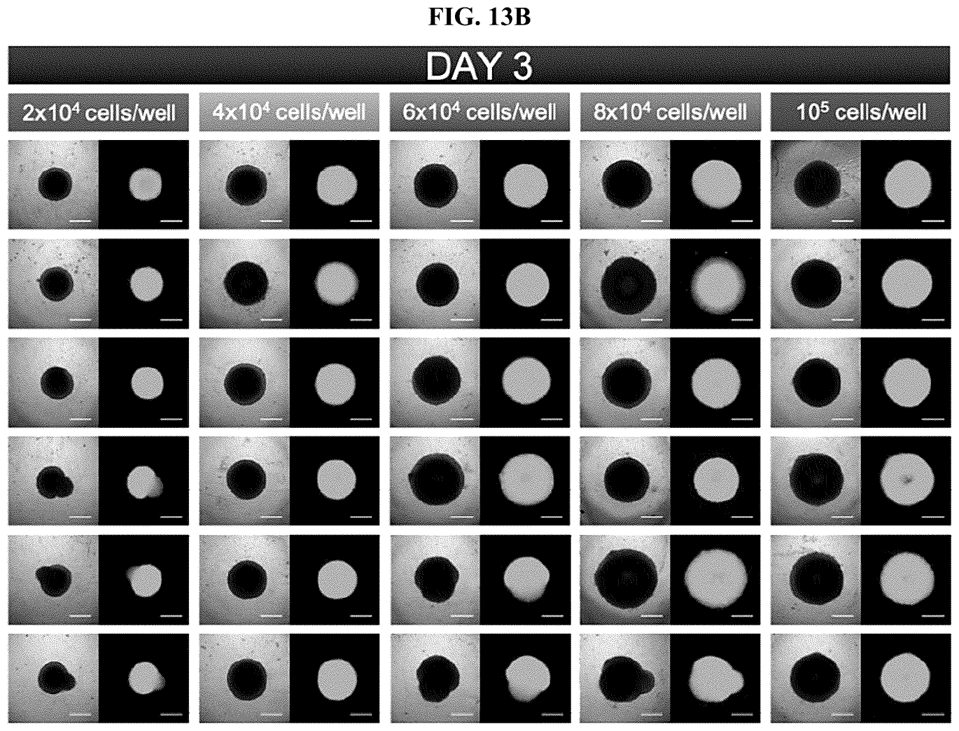

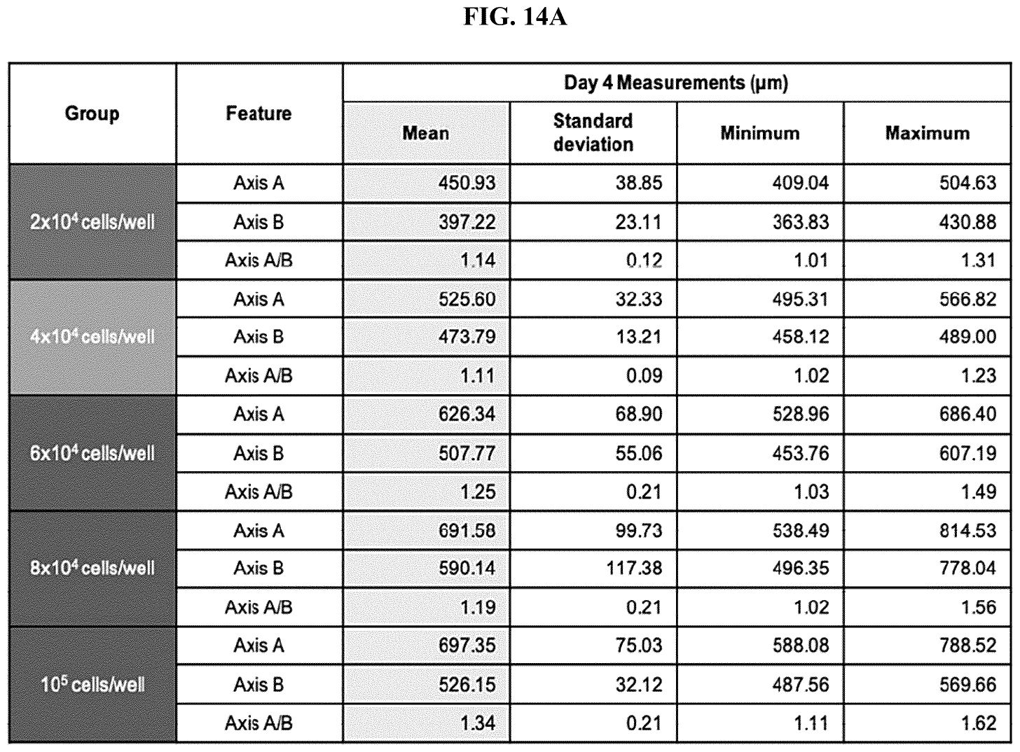

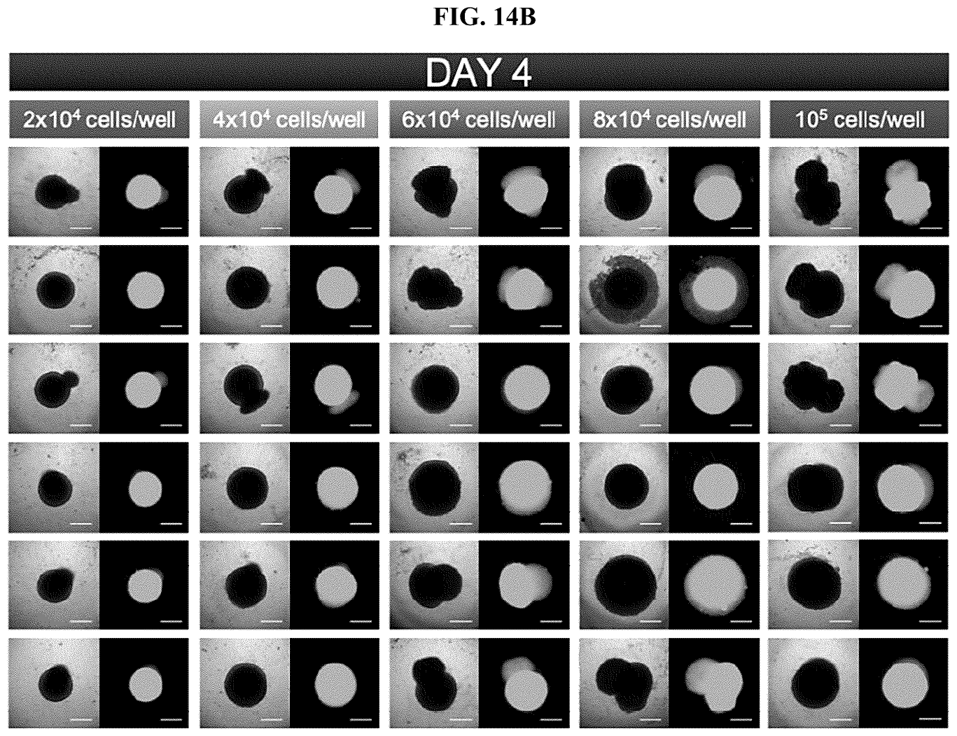

Characterization of Spheroids. Beginning 24 hours after seeding, each spheroid was imaged on an inverted Nikon Ti2 microscope, using a 4× objective and an Andor 512×512 EMCCD camera under brightfield and fluorescence using an YFP filter (for visualization of ChR2-eYFP tag) in 24-hour increments, as shown in FIG. 3A (see FIGS. 11A-11B, 12A-12B, 13A-13B, and 14A-14B for complete dataset with brightfield and fluorescent images). For smaller spheroids, such as those shown in FIGS. 3D and 3G, spheroids were imaged in the same way at days 1, 2, 5, 7, 9, 12, 14, and 16.

Morphological analysis (manual approach): Image analysis tools in NIS Elements (Nikon microscopes image acquisition software) allowed for acquisition of spatial measurements for major and minor axes using a five-point ellipse estimation on spheroid images. Size and shape were defined by equations 1 and 2, respectively, and evaluated every 24 hours.

$$radius = \frac{major axis + minor axis}{2} \qquad ( 1 )$$

$$ellipticity = \frac{major axis}{minor axis} \qquad ( 2 )$$

Morphological analysis (automated approach): Due to the rapid growth of the spheroid image datasets, an automated approach was also used for quantifying spheroid size and shape. This was achieved in MATLAB via threshold binarization and automated morphological quantification of multiple image features. Features analogous to those used in the manual approach, major and minor axes, were extracted and computed radius and ellipticity in the same way detailed above. All morphological plots shown in FIGS. 3B-3C and 3E-3Ireflect measurements obtained by this automated computational approach, though tables in FIGS. 11A, 12A, 13A, and 14A show data from manual measurements. The automated image analysis of spheroid shape was validated by the manual approach.

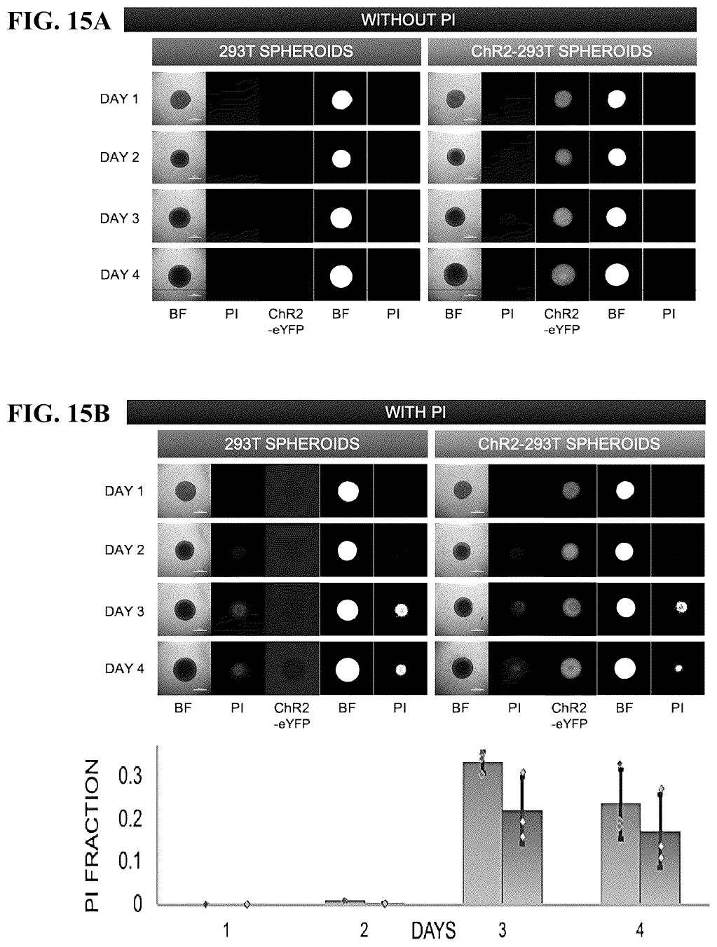

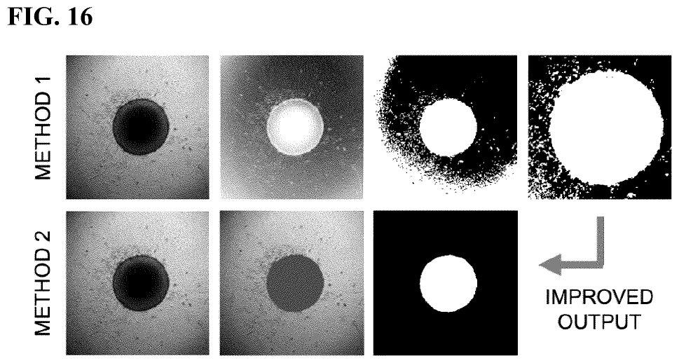

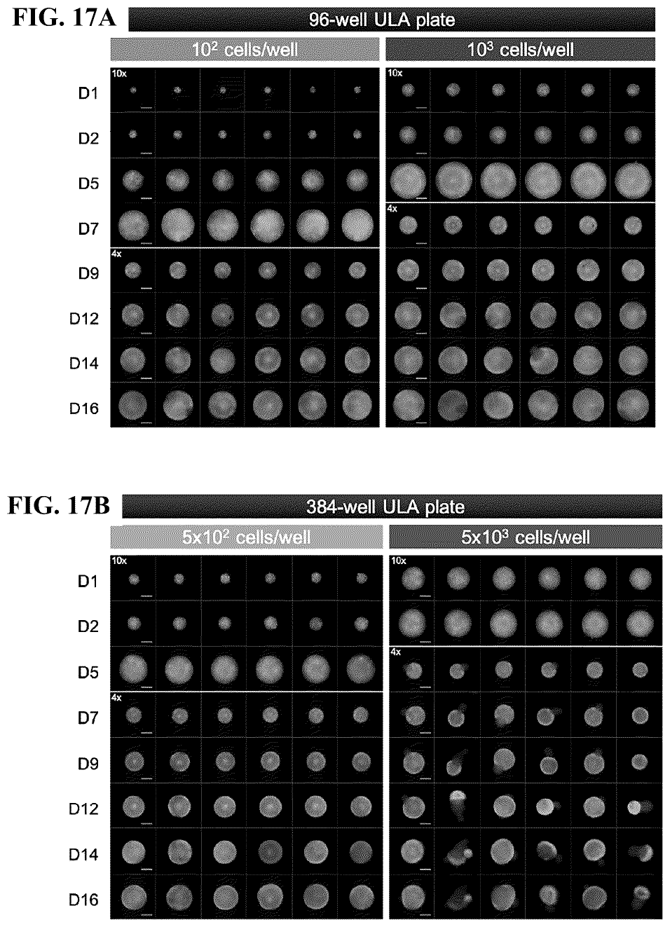

To evaluate spheroid viability, DNA-binding propidium iodide (PI) (Catalog #P3566 Thermo Fisher Scientific) was utilized. PI is impermeable to cells and thus commonly used to identify dead cells. For PI quantification experiments, WT-293T and ChR2-293T spheroids were grown in 2μg/mL PI in DMEM at 2×10⁴ cells/well. Following imaging every 24 hours, medium was replenished using the 50:50 method as detailed above, except using diluted PI instead of pure DMEM. Imaging was performed under brightfield and fluorescence to evaluate PI saturation and ChR2-eYFP expression. After normalizing acquisition parameters, the fraction of PI to spheroid area was calculated. The PI area was used as a numerator and the spheroid area from the brightfield was used as a denominator. To overcome challenges with finding an appropriate threshold to identify the silhouette of the spheroid, for the brightfield images an auto-detect ROI feature in NIS Elements was used to identify the general outline of the spheroid and register the binarization to an exported image. This direct binarization and automated ROI method for image processing are depicted in FIG. 16. Using this adjusted parameter, the fraction of PI was calculated for each image in FIG. 15B (bottom). Images of control conditions (no PI stain) for both cell lines are also included in FIG. 15A.

Culture of Stem-Cell-Derived Cardiomyocytes. Human induced pluripotent stem-cell-derived cardiomyocytes, hiPSC-CMs (Catalog #R1017, Fujifilm—Cellular Dynamics), were thawed and plated according to the supplied protocol herein. Prior to thawing, a 50 mg/mL solution of fibronectin diluted in sterile PBS was applied to all wells or plates which were then incubated overnight at 37° C. Maintenance medium was replaced every 48 hours. For initial and integration experiments, the hiPSC-CM cells were plated in 96-well glass bottom plates at 50×10³ cells/well. For macroscopic experiments, hiPSC-CMs were seeded into 14 mm glass-bottom dishes at a seeding density of 270×10³ cells/well.

Deposition of Spheroids onto Cardiac Syncytia. Channelrhodopsin-2-HEK spheroids at 2×10⁴ cells/well initial seeding density and cultured for 24 hours were transferred via pipetting onto hiPS-CM syncytia, plated 5 days before, or 7 days prior, if transfection with an optogenetic sensor was applied. For cryopreservation experiments, spheroids with initial plating density of 10² to 5×10³ cells/well were used for pacing after thawing. A wide-mouthed pipette tip with slow aspiration and deposition was utilized to minimize mechanical disturbance to the cell construct, as shown in FIG. 2B.

Immunocytochemistry. Immunocytochemistry was used to visualize the spheroid-syncytia constructs. The ChR2-expressing spheroids have an eYFP fluorescent reporter. For the hiPSC-CMs, the monoclonal anti-α-actinin antibody (Catalog #A7811, Millipore Sigma) was used to label cardiomyocyte sarcomeres and Hoechst (Catalog # H3570, Thermo Fisher Scientific) to label nuclei. After rinsing with PBS, cells were permeabilized in 0.2 Triton X-100 in 5% FBS and two-stage antibody labeling was applied to image the samples on an inverted microscope.

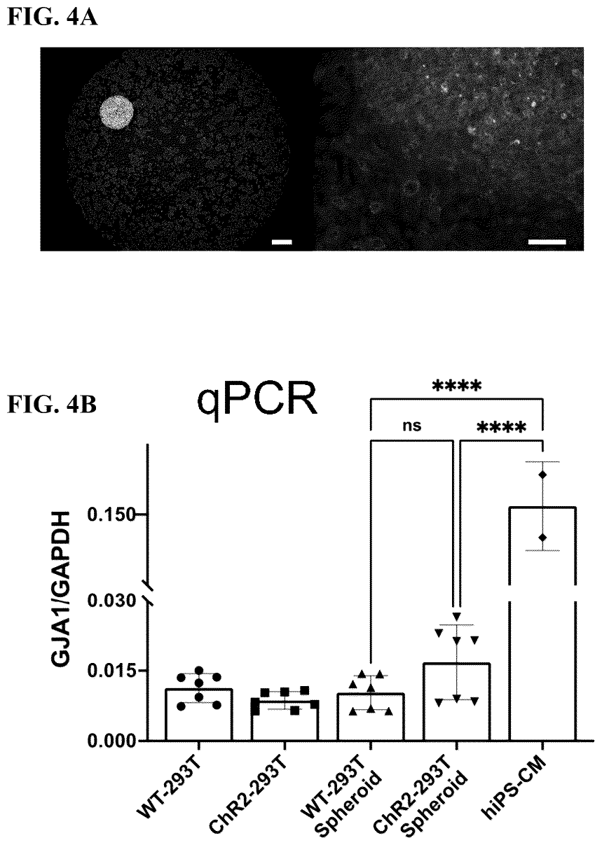

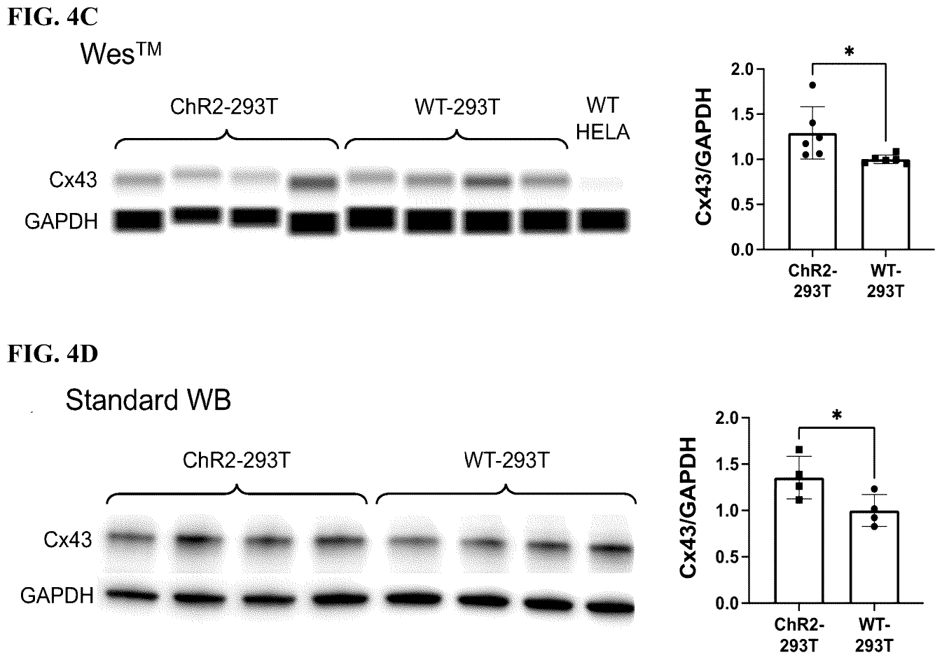

Functional Experiments Using All-Optical Electrophysiology. Functional experiments were conducted using all-optical electrophysiology using voltage or calcium small-molecule dyes or genetically encoded calcium sensors. Briefly, all functional experiments were performed in Tyrode's solution (in mM): NaCl, 135; MgCl2, 1; KCl, 5.4; CaCl2, 1.8; NaH2PO4, 0.33; glucose, 5.1; and HEPES, 5 adjusted to pH 7.4. For voltage measurements, 24-hour grown ChR2-HEK spheroids of 2×10⁴ cells per well were deposited onto hiPSC-CM syncytia in a 96-well format. After 12 hours of integration, the samples were labeled with small-molecule near-infrared voltage-sensitive BeRST1 dye at 1μM concentration. For measurements of intracellular calcium, Rhod-4AM (AAT Bioquest, Sunnyvale, CA, United States) at 10 μM or one of two genetically encoded calcium indicators (GECIs)—R-GECO or its improved version, jRGECO—expressed in the hiPSC-CMs before the deposition of the spheroids was used. R-GECO [Addgene plasmid #45494 CMV-R-GECO-1.2] or jRGECO [Addgene plasmid #61563 pGP-CMV-NES-jRGECO1a] expression was done via transfection into the hiPSC-CMs with Lipofectamine 3000 at 400 ng per sample. All microscopic measurements were done on an inverted microscope Nikon Eclipse Ti2 at 20×. Blue light 470 nm via a digital micromirror device DMD (Polygon 4000, Mightex, Toronto, ON, Canada), 5-20 ms pulses at <0.15 mW/mm 2 and using 0.5-2.0 Hz in order to trigger persistent excitation in the iPSC-CMs. Shorter pulses (down to 0.5 ms) were also used to construct strength-duration curves with a subset of spheroids. Excitation for voltage imaging with BeRST1 was at 660 nm and emitted light was collected using a long-pass filter at 700 nm, excitation for calcium imaging with Rhod-4AM, R-GECO and jRGECO was at 535 nm and emission was collected at 605 nm, in both cases with an iXon Ultra 897 EMCCD camera (Andor Technology Ltd., Belfast, United Kingdom), run at 200 fps. To avoid potential spectral overlap in the R-GECO and jRGECO measurements, patterned light was used, where the DMD stimulated region and the recorded region were physically different, as shown in FIG. 4B. The patterns for optical stimulation (DMD targeted region) and optical sensing were selected manually for an initial sample in a way so that the former included the spheroid and the latter did not. These regions were kept the same as measurements were taken from different wells in the plate. Although in the current implementation the process was not automated, it was possible to automate the spheroid positioning and then have these regions pre-programmed accordingly for HT screening.

Probing Spheroid-Syncytia Coupling. After deposition of ChR2-HEK spheroids (2×10⁴ cells/well, 24 hours) onto R-GECO-transfected hiPSC-CMs (10 days in culture including transfection with R-GECO), samples were transferred to an on-stage microscope incubator and probed for responsiveness to blue light (20 ms at 470 nm<1 mW/mm 2) pulsed at 0.5-2.0 Hz. Use of the incubator ensured temperature control at 37° C. throughout the length of the 10-12 hour experiment. Functional tests with R-GECO were performed and repeated in 2-hour increments over 10 hours for a total of 6 time-probes. This procedure allowed for determination of the emergence of system responsiveness to optical stimulation by examining the number successfully stimulated samples over time.

Macroscopic Optical Mapping. For imaging the macroscopic waves triggered by the spark-cell spheroids, the ChR2-293T spheroids (2×10⁴ cells/well, 24 hours) were deposited onto hiPSC-CM samples plated in 14 mm glass-bottom dishes after 24 hours of integration. The widefield optical mapping of cardiac excitation wave was built around an MVX10 MacroView, Olympus, Japan system. A high-speed CMOS camera (Basler, Ahrensburg, Germany) was used to track wave dynamics. Either Rhod-4AM or R-GECO were used as calcium sensors for these measurements. Continuous fluorescence excitation light with an irradiance of 0.28 mW/mm 2 was generated by a collimated green LED (M530L3, Thorlabs, United States), which obliquely illuminated the hiPSC-CMs dish from underneath. Pulsed optical pacing light with an irradiance of 0.35 mW/mm 2 was generated by a weakly focused LED (M470L4, Thorlabs, United States), which was driven at 0.5 Hz (or 1 Hz) with a pulse duration of 10 ms. Microscopic and macroscopic recordings were processed using Matlab software for filtering and visualization.

Gene Expression Analysis by qPCR. Cells were plated on 96-well format. Two days post plating, cells were harvested for RNA extraction and mRNA levels were detected and quantified using Power SYBRTM Green Cells-to-CTTM Kit (Cat. 4402953, Invitrogen) according to the manufacturer's protocol. qPCR analysis was performed on a QuantStudio 3 Real-Time PCR System (Thermo Fisher Scientific) with the QuantStudio Design and Analysis Software (Thermo Fisher Scientific). Quantification of gap junctional gene expression of GJA1, encoding for Cx43, (primers: Fw GGTGGTACTCAACAGCCTTATT (SEQ ID NO: 1); Rev ACCAACATGCACCTCTCTTATC (SEQ ID NO: 2)) was normalized to expression of housekeeping gene GAPDH (primers: Fw GGAGCGAGATCCCTCCAAAAT (SEQ ID NO: 3); Rev GGCTGTTGTCATACTTCTCATGG (SEQ ID NO: 4)) using a standard AACt method.

Protein Quantification. Cells were lysed for total protein in 96-well format using the Qproteome Mammalian Protein Prep Kit (Cat. 37901, Qiagen). Protein lysates were loaded onto and analyzed using either traditional gel electrophoresis-based western blot or the Wes (ProteinSimple) capillary-based system for protein quantification. The Wes system allows for protein quantification in small samples in a semi-automated way; more details on both methods can be found in Li, Han, and Entcheva, Am. J. Physiol. Heart Circ. Physiol. 319, H1112—H1122 (2020), the disclosure of which is incorporated herein in its entirety. Proteins of interest were probed using antibodies specific to Cx43 (ab11370, Abcam) and GAPDH (ab181602, Abcam). Changes of Cx43 protein levels were presented as normalized to GAPDH.

Spheroid Cryopreservation for Long-Term Storage. Spheroids grown in ultra-low-adhesion microplates (96 well and 384 well) were optimized by size for purposes of freezing/thawing. Smaller spheroids (with <10⁴ cells at plating; initial diameter <0.3 mm) performed better when frozen and thawed. After optimization, freezing medium with the following composition (in %) was utilized: DMSO:DMEM:FBS (10:30:60). After 24 hours of plating in the ULA microplates (96 well or 384 well), spheroids were transferred to this freezing medium in cryovials. First, controlled cooling 1° C./minute was applied overnight in a 31 80° C. freezer. After that, the cryovials with spheroids were transferred to liquid nitrogen (−120° C.) for long-term storage (7-10 days in this study). Quick thawing (using a 37° C. water bath) was applied and the spheroids were transferred to DMEM culture medium in 96-well ULA microplates. Propidium iodide (2 μg/ml) was added to monitor viability. Spheroids were recovered for 4 days and then administered to monolayers of human iPSC-CMs in 96 well format. Functional testing (using optogenetic sensor jRCEGO for intracellular calcium and blue pulses of light for pacing) was done within 18 hours after depositing the spheroids.

Statistics. All statistical analysis was performed in GraphPad Prism. Changes in spheroid morphology over time and initially seeding density was evaluated for significance via two-way ANOVA with post-hoc Tukey or Sidak. Differences in gene expression between assessed groups was tested for significance via one-way ANOVA with post hoc Tukey. Finally, for protein quantification data by Wes and standard western blot, an unpaired t-test was used. In all statistics, p<0.05 and normality of the data was tested via Shapiro—Wilk test.

### Example 2

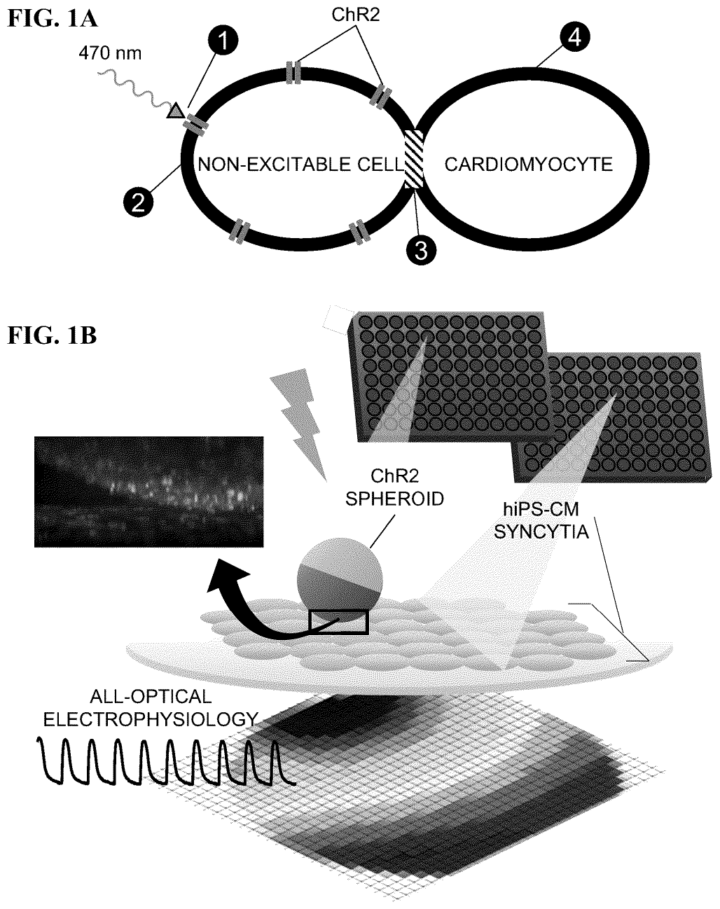

Exemplary methods herein demonstrated the manufacturing and characterization of “spark-cell” spheroids. In brief, the goal in manufacturing of spark-cell spheroids was to scale up the so called “tandem-cell-unit” (TCU) approach, as depicted in FIG. 1A, into an easy-to-handle, configurable three-dimensional structures of ChR2-expressing cells for use in HT drug screening of cardiac tissue. Such “spark-cell” spheroids composed of optogenetically transformed live cells can be positioned atop hiPSC-CM syncytia, as shown in FIG. 1B, to mediate optical pacing.

To form three-dimensional cell constructs, spheroid formation via self-aggregation was used in the exemplary methods herein due to the method's simplicity. Cells deposited in agarose molds can self-assemble into three-dimensional tissue spheroids. ChR2-HEK spheroids were fabricated in commercially available 96-well or 384-well microplates with ultra-low attachment treatment, as shown in FIG. 2A. The assembly was further facilitated by gravity. It was possible to use wide-mouth pipette tips to handle these spheroids, i.e., to carefully lift, transfer, and to deposit them on top of hiP SC-CM monolayers (grown in 96-well plates), as shown in FIG. 2B.

Optimization of seeding cell density included considerations for easy handling (the spheroids had to be large enough) and avoiding the formation of a significant necrotic core (not too large). Initial seeding density between 10⁴ and 10⁵ cells/well yielded viable and functional spheroids recognizable by the human eye and therefore easy to handle. However, with optimization of spheroid handling, it was possible to generate spheroids of smaller seeding density, on the order of 10² to 10³ cells/well in both 96-well format as well as in 384-well format.

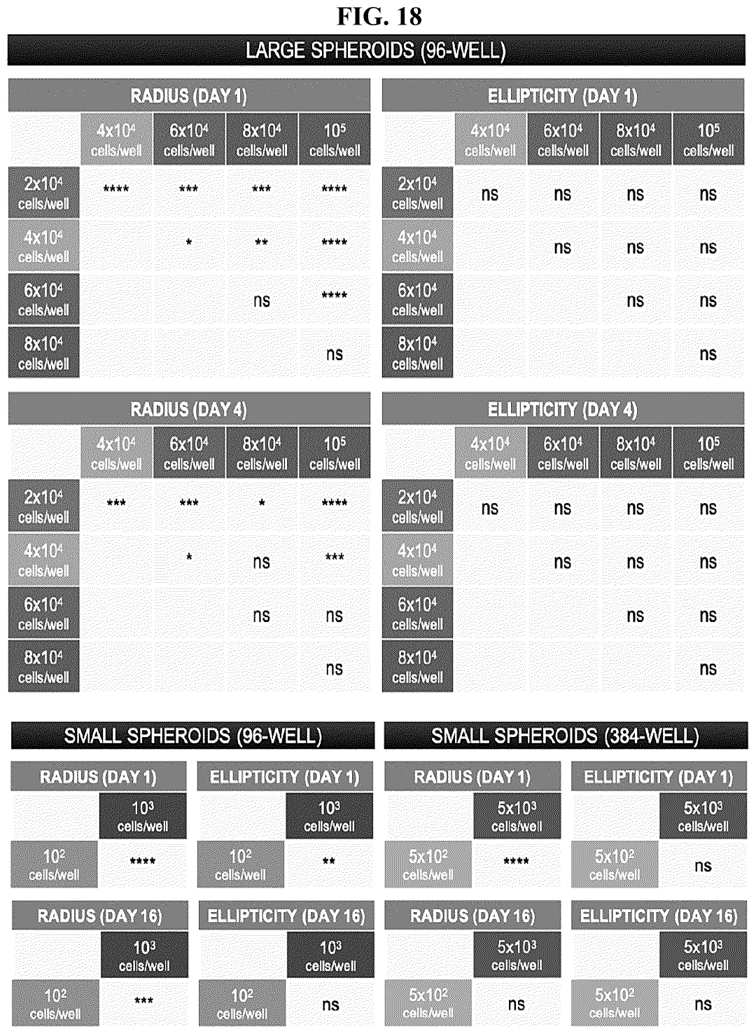

Once formed, exemplary methods herein characterized the spheroids. In brief, larger spheroids, formed using initial seeding densities from 2×10⁴ to 10⁵ cells/well, in increments of 2×10⁴ cells/well were explored in the methods herein. To register how spheroids evolved structurally over time, brightfield images were captured every 24 hours, along with images of eYFP to monitor the expression of ChR2-eYFP (FIG. 3A). For morphological analysis of the spheroids, threshold binarization and morphological operations was applied on the images to obtain quantitative parameters that could be used to describe the observable evolution of size and shape (ellipticity) (FIGS. 3B-3C). Large spheroids at the studied seeding densities became more compact over the first 48 hours, then grew in size due to cell proliferation. A two-way ANOVA revealed that spheroid size was significantly impacted (p<0.05) by both time of culture and seeding density. For these larger spheroids, ellipticity was not significantly impacted by seeding density but only by time in culture (p<0.05), where at day 4, all groups showed significant deviations from a perfect sphere (captured here by increase in ellipticity). A subset of the statistics (pairwise comparisons using post hoc Tukey correction) is shown in FIG. 18.

Characterization of smaller spheroids was undertaken later in the study, as spheroid handling procedures were optimized. It was reasoned that a smaller seeding density would allow better consistency of shape and size over time, leading to a longer window of spheroid usability and higher viability for storage applications. Shown in FIG. 3D are smaller-size spheroids at seeding density 10² and 10³ cells/well in 96-well plates at days 1 and 5. In the corresponding 16-day morphological analysis shown in FIGS. 3E-3F, a steady increase in radius was observed but relatively little deviation from a perfect sphere was seen. A two-way ANOVA showed significant dependence of spheroid size both on seeding cell density and on time in culture (p<0.05); ellipticity, similar to the larger spheroids, was only influenced on time in culture but not on seeding density. Overall, these spheroids showed much lower deviations from a sphere over more than 2 weeks in culture. Spheroids were also manufactured in 384-well plates; samples at 5×10² and 5×10³ cells/well are shown at days 1 and 5 in FIG. 3G. In this format, spheroid radius initially grew and then reached a size limit, as shown in FIG. 311, likely due to the higher curvature of the well. Similar to the larger spheroids (>10⁴ cells/well), after several days in culture, the ellipticity of the 5×10³ cells/well group (FIG. 31) increased substantially. All groups of smaller spheroids (up to 10³ cells/well) did not demonstrate any stages of compression, but rather steadily grew in size while maintaining perfect spherical shape over 2 weeks in culture (FIG. 3E-3F). Applying a two-way ANOVA, it was found that spheroid size and ellipticity were influenced significantly (p<0.05) by seeding density and time in culture. Pairwise comparisons are shown in FIG. 18. Overall, for a known plating cell density, spheroid size was highly reproducible as shown by the tight distributions in FIGS. 3A-3I, and these smaller spheroids were more versatile as they remained intact and close to a sphere within over 2 weeks.

In the large spheroids, the viability of the constructs was also assessed by culturing in DMEM containing PI at 2 μg/mL. Every 24 hours, cells were imaged and medium replaced with the same PI dilution. This characterization experiment generated the images for FIGS. 15A-15B, in which WT 293T and ChR2-infected 293T spheroids were seeded at 2×10⁴ cells/well monitored over time. Control group data (spheroids in PI-negative culture medium) in FIG. 15A is shown alongside experimental group data (spheroids in PI-positive culture medium) in FIG. 15B. Binarization of the spheroids with PI images revealed a quantitative PI fraction for each cell line and day (n=3 for each condition), summarized at the bottom of FIG. 16. Spheroids developed a necrotic core with time in culture as shown by the localization of PI in the center of the structures. However, these necrotic cores were surrounded by a wall of viable cells, therefore this did not affect their acute use to pace cardiomyocytes.

### Example 3

Exemplary methods herein demonstrated a relationship between gap junctions and the “spark-cell” spheroids. Immunocytochemistry and confocal imaging helped visualize the coupling of the spheroids with cardiomyocyte layers (FIG. 4A). Of interest was the coupling mechanism between the ChR2-spheroid and hiPSC-CM syncytia in this larger-scale TCU approach. Gap junctional proteins, such as Cx43 (abundantly expressed in ventricular cardiomyocytes), were thought to play a role, even though the HEK cells are known to have minimal amounts of Cx43. Small-sample qPCR and protein quantification assays were used to probe for GJA1/Cx43 expression levels, normalized by GAPDH transcript/protein, respectively. As expected, the lysed monolayers and spheroids of WT-HEK and ChR2-HEK cells had an order of magnitude lower GJA1 mRNA levels (normalized to GAPDH) compared to the human iPSC-CMs, p<0.05 by one-way ANOVA and Tukey post hoc comparisons (FIG. 4B). Yet, the spheroids had detectable, albeit low, levels of GJA1. Furthermore, the ChR2 expression seemed to slightly increase GJA1 mRNA (n.s.), n=7 biological replicates per group (with 3 technical replicates per sample). At the protein level (FIGS. 4C-4D), ChR2-HEK also showed some increase in normalized Cx43 protein levels compared to the WT HEK. Specifically, unpaired t-test in standard western blot results, showed that ChR2-HEK had 35% higher Cx43/GAPDH levels compared to WT-HEK (p=0.048, n=4 biological replicates per group); in Wes runs, about 41% increase was seen (p=0.035, n=6 biological replicates per group). The presence of some Cx43 in the “spark-cell” spheroids suggests gap junctional contribution to their integration with the human iPSC-CMs, although other mechanisms could be at play as well.

### Example 4

Exemplary methods herein assessed functionality of the spheroids. To optimize the stability of the larger spheroids, it was determined that the most impactful constraints were early time in culture and low initial seeding density, e.g., 2×10⁴ cells/well. Hence, functional testing experiments were performed at this cell density after 24 hours upon depositing the spheroids onto 96-well monolayers of hiPSC-CMs (pre-cultured for 5 days post-thaw) as described in FIG. 1B.

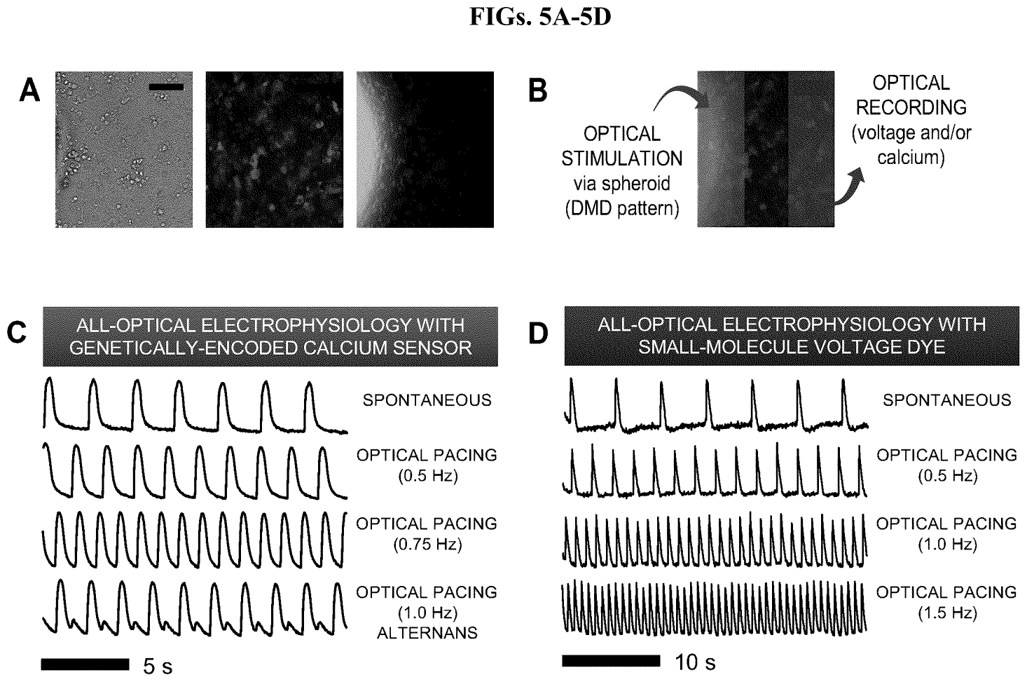

Typical all-optical electrophysiology experiments (as in FIGS. 5A-5D) were done 12 hours post spheroid deposition onto hiPSC-CMs grown in 96-well plates. For optical confirmation of pacing: 1) two genetically-encoded calcium sensors (GECIs), R-GECO or jRGECO, expressed in the iPSC-CMs; or 2) a near-infrared voltage-sensitive dye, BeRST1 were used. Both methods were spectrally compatible with the ChR2 actuator. To further suppress potential cross-talk when using calcium recordings with R-GECO or jRGECO, the light was spatially patterned to confine the excitation near the spheroid and optically recorded from a nearby region (FIG. 5B). Such restrictions were not necessary when using the red-shifted voltage-sensitive dye.

FIGS. 5C-5D show traces of intracellular calcium (via R-GECO) and action potentials (via BeRST) from spontaneous and paced activity via “spark-cell” spheroids. Successful pacing was confirmed when samples followed 1:1 the selected frequencies over at least 10 consecutive beats. Incomplete capture sometimes resulted in alternans or 2:2, as shown in FIG. 5C. Note that the maximum capture frequency was strongly dependent on temperature. In these experiments, performed at 25-30° C., typical maximum capture frequency was between 0.7 and 1.5 Hz. These responses were consistent with electrical pacing.

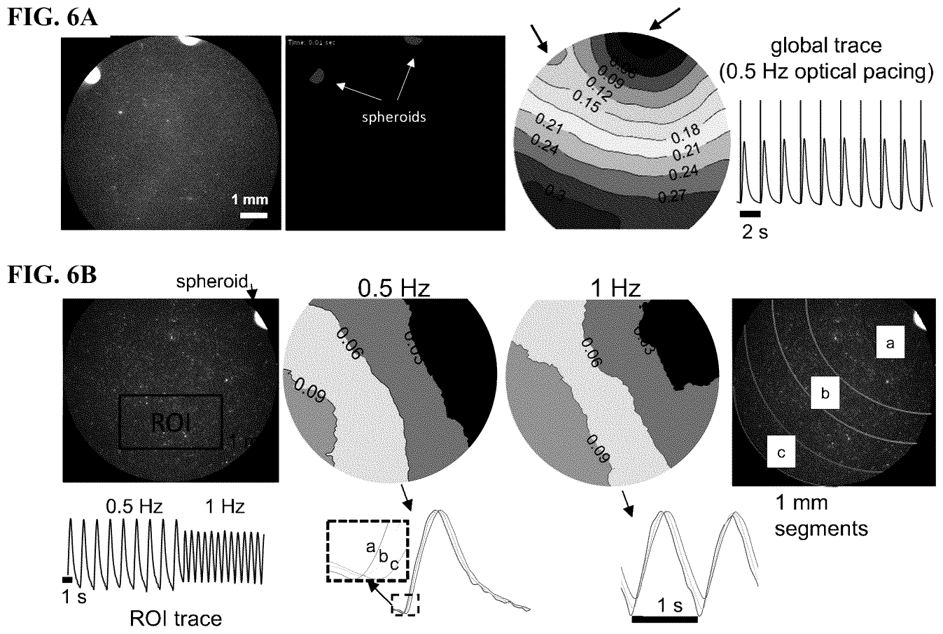

To confirm that the “spark-cell” spheroids were capable of pacing cells in larger cm-scale cardiac samples and to map the waves triggered by the spheroids, some macroscopic optical mapping experiments were also performed in 14 mm dishes (FIGS. 6A-6B). For these experiments, the spheroids were positioned on top of the cardiomyocytes 24 hours before the imaging. Spheroid-triggered excitation waves were registered using either R-GECO or a Rhod4-AM calcium sensor, as shown in FIGS. 6A-6B. Light was aimed at the spheroids but not focused on them. When more than one spheroid was used, as in FIG. 6A, both initiated activity locally (black arrows) upon light trigger but a dominant pacemaker emerged, driving the cardiac syncytium on each beat.

### Example 5

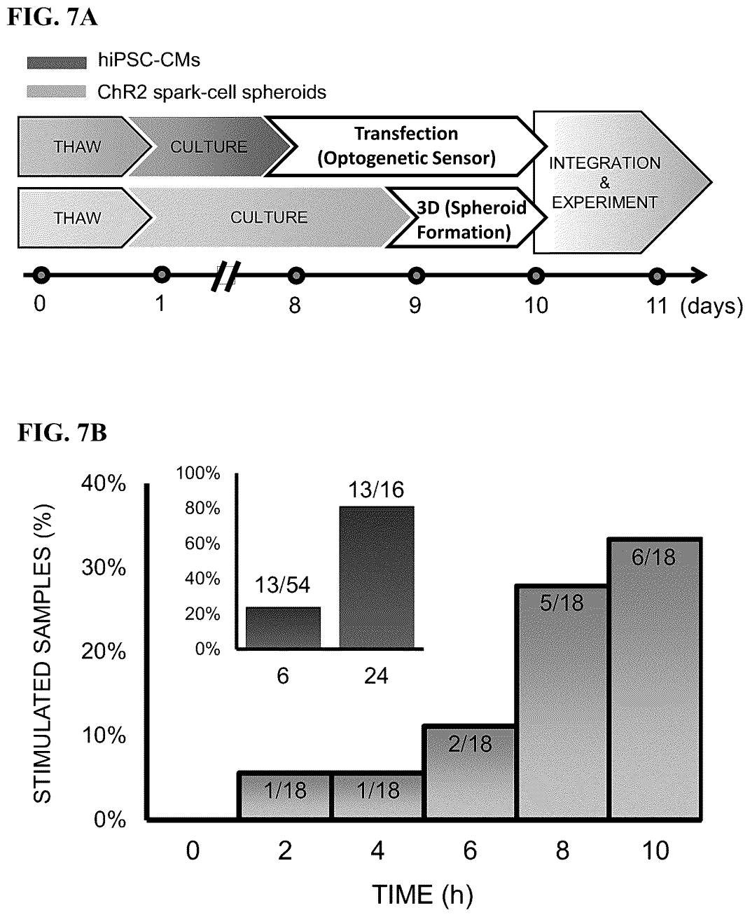

Exemplary methods herein assessed the timing of integration. In brief, after confirming spheroid functionality, the earliest emergence of TCU-based coupling was probed for in the system detailed herein. To do so required the approximate timeline of experimental preparation shown in FIG. 7A.

In an iPSC-CM culture of sample size of n=18 biological samples, measurements were performed every 2 hours over a 10-hour window using on-stage incubator with temperature control to minimize potential mechanical disturbance from transferring samples to and from an external incubator and imaging system. In this culture, 1 out of 18 samples showed responsiveness to optogenetic pacing after only 2 hours with an increasing number of samples coupling over the experiment. At the end of 10^{hours,} 6 of the 18 samples showed responsiveness to optogenetic pacing. These results are shown in FIG. 7B. Separate less involved experiments were conducted to capture integration efficiency at just 6 hours and at 24 hours post-spheroid introduction. Five of the 16 biological samples in this iPSC-CM set, were successfully paced optogenetically at 6 hours; and 13 out of the 16 samples were responsive at 24 hours. To increase sample size just at the 6-hour time point, the same responsiveness efficiency was tested at 6 hours post-spheroid introduction for additional 20 iPSC-CM cultures, for which 6 out of 20 samples demonstrated optical pacing at 6 hours.

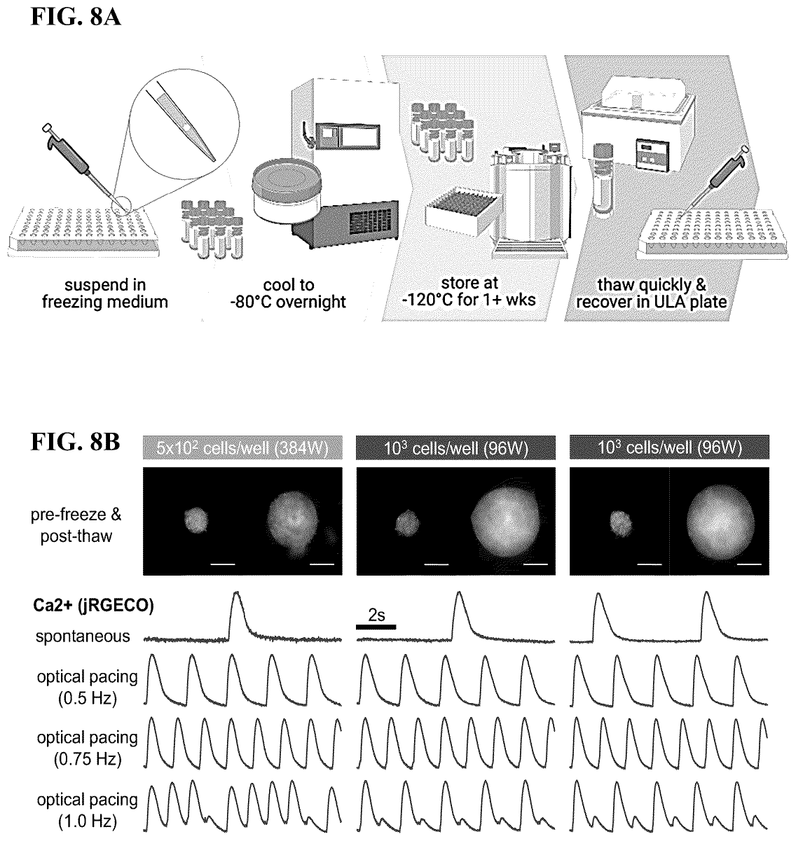

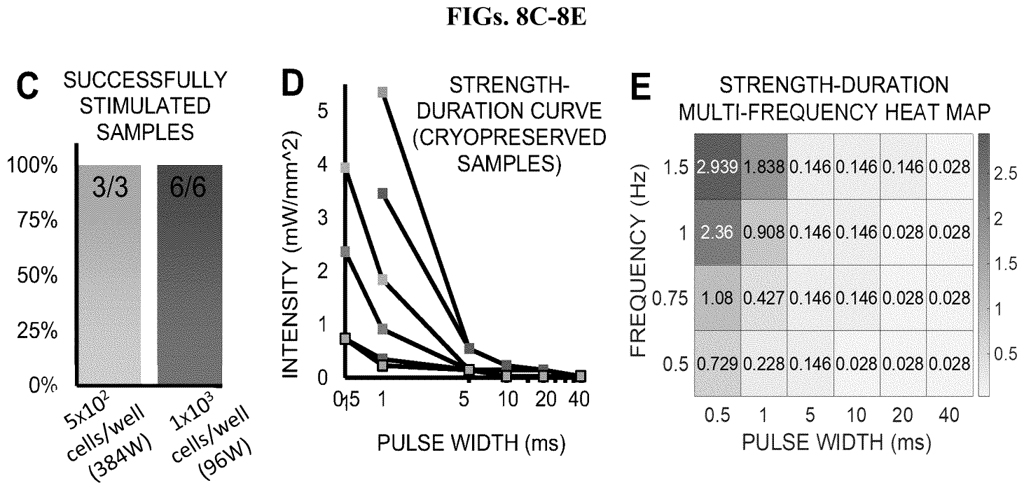

In total, experiments monitoring timing of integration over several iPSC-CM cultures were conducted for a total combinable dataset of n=54 independent samples with various configurations of tested time points. These data demonstrate high efficiency of the “spark cell” spheroids to couple with cardiac syncytia, as 13 out of 16 (81%) iPSC-CM cultures (first subset only) yielded responsiveness after 24 hours, and overall 13 out of the entire 54 (24%) iPSC-CM cultures (all three subsets) demonstrated functionality after only 6 hours post-introduction of the spheroid to the cardiomyocytes. This summary data is shown in the inset of FIG. 7B. Note that with optimization of the handling technique, 100% integration was achieved for the cryopreserved and thawed spheroids at 18 hours post-deposition (see FIGS. 8A-8E). The early emergence of optogenetic pacing confirmed by these experiments confirmed that the “spark cell” spheroid approach disclosed herein was able to yield functional optical pacing significantly earlier than the standard direct viral or liposomal transductions of the cardiomyocytes, which typically require 48 hours.

### Example 6

Exemplary methods herein examined spheroid cryopreservation and successful deployment after thawing. Of interest for this modular approach to contactless pacing was whether the spheroids could be used as “reagents” that can be stored, transported and deployed on demand. As long-term storage and transportation through cryopreservation has been a commonly used technique for cell suspensions, a similar approach to preserving these multi-cellular structures was applied similar to that described in Kim et al., PLoS One. 13:e0196714 (2018), the disclosure of which is incorporated herein in its entirety. In brief, spheroids (on the order of 10²-10³) were frozen in a freezing medium consisting of 30% DMEM +60% FBS +10% DMSO in a controlled manner to ˜80° C. overnight. The following day, samples were transferred to liquid nitrogen at ˜120° C. for 1 week. Subsequently, the samples were then successfully thawed and recovered in a 96-well spheroid microplate. This cryopreservation workflow is shown schematically in FIG. 8A.

After the first day post-thaw, spheroids were labeled with PI diluted in medium for viability analysis, where medium was replaced every 48 hours. FIG. 8B shows three successfully recovered spheroids 1 day before freezing (left) and 6 days after thaw from liquid nitrogen (right). Of these three depicted samples, 1 spheroid had been seeded at 5×10² cells/well in a 384-well spheroid plate while the other two spheroids had been seeded at 10³ cells/well in a 96-well spheroid plate. Though PI staining (red) revealed a small amount dead tissue on the surface of one of the samples, this did not affect the spheroids ability to couple with the cardiomyocytes. Indeed, all three depicted samples successfully elicited calcium transients as recorded by jRGECO in cardiomyocytes, with spontaneous as well as light-controlled pacing at different frequencies: 0.50 Hz (1:1 response), 0.75 Hz (1:1 response), and 1.0 Hz (alternans). In total, three spheroids at 5×10² cells/well in a 384-well format and six spheroids at 103 cells/well in a 96-well format were successfully thawed and demonstrated spheroid functionality in nine cryopreserved samples, as depicted in FIG. 8C. Note that all nine out of nine tested samples achieved successful optical pacing within 18 hours post-deposition.

From the six additional samples tested (not the 3 shown in FIG. 8B), individual strength-duration curves were constructed (FIG. 8D) to assess the blue light intensity and pulse width thresholds necessary for 1:1 coupling. As stated in the description for functional experiments, 20 ms pulses at very low light intensity (<0.1 mW/mm²) was sufficient for optical pacing, with higher intensities at shorter pulse widths, down to 0.5 ms. Note that the six samples needed quite different light levels when the pulses become very short. Using fibroblasts and cardiomyocytes, Boyle et al., Sci Rep. 29;11(1):9310 (2021), the disclosure of which is incorporated in its entirety herein, showed experimentally and computationally that the irradiance needed to trigger optical pacing (at short pulses, in particular) can effectively be used to quantify coupling in such assemblies.

For one of the six samples assessed in FIG. 8D, the strength-duration threshold was further explored at multiple frequencies. The data for this particular sample is shown in FIG. 8E as a heat map, where blue light intensity in mW/mm 2 was a function of pulse widths (0.5, 1, 5, 10, 20, and 40 ms) and frequency (0.5, 0.75, 1.0, and 1.5 Hz). This data indicated >1 mW/mm 2 intensity requirement for only four of the frequency-to-pulse width paired parameters, involving particularly short pulses of 0.5 and 1 ms.

Overall, the demonstration of functionality with spheroids that have undergone cryopreservation herein suggests that these “spark-cell” spheroids can be treated as transportable reagents that are storable and deployable on demand for on-site optical cardiac pacing.

### Example 7

Exemplary methods herein generated small tissue structures (spheroids, made of 100 to 10,000 cells, diameter of 0.15 to 1 mm), endowed with the ability to respond to electrical, optical, magnetic and/or ultrasound/mechanical actuation that could be cryopreserved and deployed on demand for multimodal stimulation of muscle or as building blocks for soft robotics. Four modalities contemplated herein for use in a single cellular structure included the following: 1) electrical actuation that was enabled by using excitable cells or by genetically creating such, e.g. the “Spiking-HEK” cell line with Kir2.1 and Nav1.5 gentically expressed; 2) optical actuation that was enabled in the cells by genetic expression of opsins (e.g., ChR2); 3) magnetic actuation that was enabled by loading magnetic nanoparticles (e.g., 100 nm dextran iron-oxide composite); and 4) mechanical actuation, i.e. responsiveness to ultrasound, fluid flow, pressure puffs and other mechanical forces that was enabled by genetically expressing a mechanosensitive ion channel, Piezol. Of these four modalities, one or more—including all four—can be used in the cells used to generate the spheroids disclosed herein.

In an exemplary method, magnetic nanoparticles can be loaded into the spheroids disclosed herein (e.g., spark-cell spheroids). In brief, cross-linked dextran iron oxide composites (Micromod, Germany), 100 nm, 5 mg/ml, with red fluorescent tag (ex: 552, em: 580) were used. These magnetic nanoparticles (MPs) were used in conjunction with a constant magnet plate (from Takara). The steps of the exemplary method were performed as follows: 1. Grow ChR2-HEK293T in 48-well glass-bottom plate to 90% confluency; 2. MPs aliquoted into 1 ml stock and sonicated for 10 minutes; 3.MPs—mixed with serum-free DMEM using serial dilution for concentrations ranging from 1 μg/ml, 5 μg/ml, 10^{μg/ml,} 50 μg/ml, 100 μg/ml, 500 μg/ml, 1 mg/ml; 4. Sonicate the prepared solutions again; 5. Aspirate the culture medium from the cells and apply the prepared MP-containing solutions (1 ml per well); 6. Incubate for 2 hours at 37° C. in the incubator while positioning the plate on the magnet; 7.(optional) Image the samples at 40× using the mCherry cube to document the nanoparticles in the monolayers—after 1 hour of incubation, endocytosis should have taken place; 8. After incubation, wash samples 2× with PBS; 9. Trypsinize with 0.25% Trypsin EDTA until cells are dissociated; 10. Add DMEM with 10% FBS to samples to block trypsin; 11. Count cells and dilute to achieve solution to plate 1000 cells per well in a 96-well ULA plate to form spheroids; 12. Incubate the spheroids for 24 hours and image using filters for the ChR2-eYFP and for the MP-red-tag (mCherry); 13. The MP-ChR2-spheroids are ready to be deposited onto cardiac monolayers for functional testing or to be cryopreserved. Actuation of the MP-containing spheroids were performed by the magnetic plate or, alternatively, by more sophisticated magnetic arrangements. Beyond quick spheroid “clamping” to the bottom (to the cardiac layer/tissue), magnetic navigation provides more intricate x-y-z guidance. The “magnetic actuation” is a positioning force, unlike the electrical, optical and mechanical actuation, which all involve some ion transport event that then alters the electrical potential of the entity to which the spheroid is coupled. The magnetically-controlled positioning herein is useful to accelerate coupling by “clamping” the spheroid down to the muscle construct. In a more general sense, it is useful for x-y-z positioning control and can be critical for quick assembly.

In another exemplary method, ultrasound/mechanosensitive ion channels were genetically expressed in the spheroids disclosed herein (e.g., spark-cell spheroids). First, an adenoviral vector carrying the human Piezol (pAd-CMV-hPIEZO1) was developed. Spheroids expressing Piezol were prepared according to the steps of the following method: 1. Grow ChR2-HEK293T in 48-well glass-bottom plate to 70% confluency. These can be loaded with MPs as described above by 2 hour incubation; 2. Prepare AdV solution of 50 MOI in serum-free DMEM; 3. Apply to the ChR2-HEK293T for 2 hours at 37° C. incubator, agitating periodically; 4. Remove the virus-containing solution and replace with DMEM with 10% FBS; 5. Expression should be present in 48 hours. Confirmation by qPCR after lysing some of the samples; 6. After 48 hour incubation, wash samples 2p33 p9 with PBS; 7. Trypsinize with 0.25% Trypsin EDTA until cells are dissociated; 8. Add DMEM with 10% FBS to samples to block trypsin; 9. Count cells and dilute to achieve solution to plate 1000 cells per well in a 96-well ULA plate to form spheroids; 10. Incubate the spheroids for 24 hours and image using filters for the ChR2-eYFP (mCherry cube for MP-red tag); reserve some spheroids for qPCR analysis to confirm hPiezol; 11. The hPiezo1-(MP)-ChR2-spheroids are ready to be deposited onto cardiac monolayers for functional testing or to be cryopreserved.

Actuation of the hPIEZO1 is performed, for example, by 0.5 to 1W/cm² ultrasound transducer using 0.5 to 1 kHz. Additionally, small molecule Yoda-1 is used as an agonist to make the mechanical actuation of the ion channel easier, because Yoda-1 lowers the hPIEZO1 channel's threshold for mechanical activation. The result of ultrasound engagement of Piezol yields similar effects to the optogenetic actuation of ChR2.

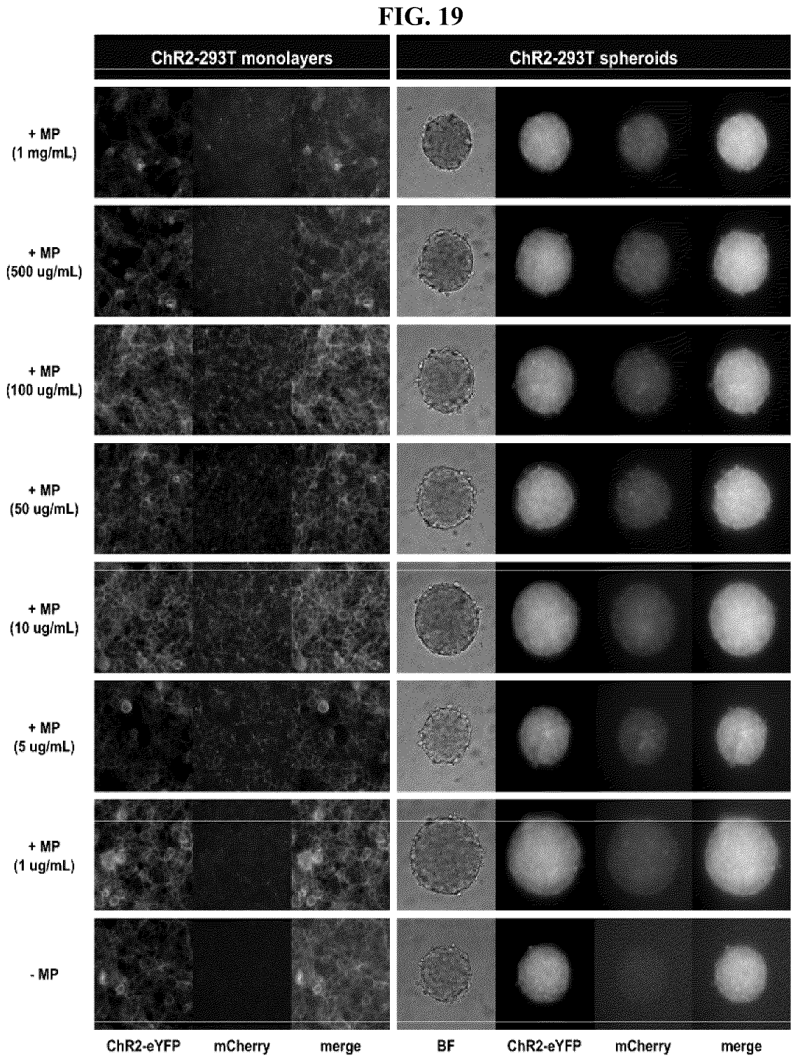

FIG. 19 shows spheroids formed from ChR2-HEK293T cells expressing Piezol loaded with magnetic nanoparticles according to the exemplary methods detailed herein.

The spheroids formed from ChR2-HEK293T cells expressing Piezol loaded with magnetic nanoparticles were grown in ultra-low-adhesion microplates (96 well and 384 well) where spheroid size was optimized for purposes of freezing/thawing. The spheroid diameter at freezing was approximately 200 μm. Special freezing medium was used as follows: DMSO:DMEM:FBS (10:30:60%). Spheroids were stored in this freezing medium in cryovials. First, controlled cooling 1° C./min was applied overnight in Nalgene Mr. Frosty container with isopropyl alcohol in a −80° C. freezer. After that, the cryovials with spheroids were transferred to liquid nitrogen (−120° C.) for long-term storage.

Quick thawing method in a 37° C. water bath was applied followed by quickly removing ice crystals by pipetting within minutes from insertion of the cryovials in the bath, and spheroids were transferred to DMEM:FBS (98:2%) culture medium in 96-well ULA microplates. Propiduim iodide was applied at 2 μg/ml to monitor viability. Spheroids were recovered for 4 days and then administered to monolayers of human iPSC-CMs in 96 well format. Functional testing (using optogenetic sensor jRCEGO for intracellular calcium and blue pulses of light for pacing) was done within less than 24 hours after depositing the spheroids. All thawed spheroids prepared this way were successfully mediating optical pacing. The smallest spheroids were those with 100 cells, grown over 24 h prior to freezing.

Biophotonic device analysis was performed with co-culture of cells or with sequential culture of cells. The formation of a defined 3D structure had the following distinct benefits: amenable to robotic handling and automation for precise deposition; reproducible location-specific pacing; lowest energy needed to pace (very low light levels); controlled manufacturing, freezing/thawing/storage—a “reagent” that can be commercialized in some form and easy to apply; and, with 3D printing, one can potentially manufacture other shapes of 3D structures to be handled the same way.

### Example 8

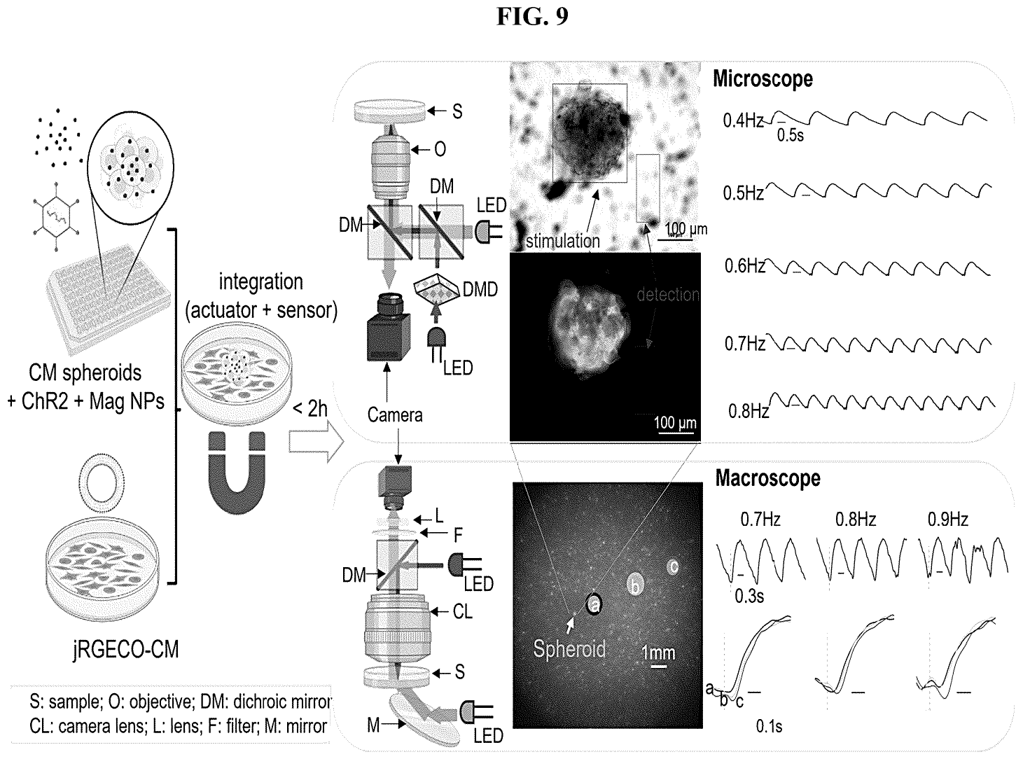

Exemplary methods herein assessed magnetically-mediated fast navigation of optogenetic spheroids. In brief, cardiomyocyte cell spheroids were assembled in U-shaped 96-well plates using human iPSC-CMs (FIG. 9, top left). Cells were virally transformed to express an optogenetic actuator, ChR2, upon plating. Magnetic nanoparticles (0.2 μm) were administered after spheroid formation. Optogenetically and magnetically-active cardiac spheroids were made of approximate size 0.2 mm in diameter. In parallel, iPSC-CMs were grown in glass-bottom 35-mm dishes and were transformed to express an optogenetic sensor, e.g. jRGECO, to report changes in intracellular calcium (FIG. 9, bottom left).

After day 5, an optogenetically- and magnetically-active cell spheroid was deposited onto each 35 mm dish and the dish was positioned onto a magnetic plate to pull down the spheroid for quick integration with the cardiomyocyte layer. In less than 2 hours the integration of the cell spheroid with the cell monolayer was confirmed through functional measurements using microscopic and macroscopic recordings as shown with custom-built systems. Blue light was used to pace the ChR2-expressing spheroids with variable pacing frequency from 0.4 Hz to 1.2 Hz. The response of the cell monolayers (not expressing ChR2) was recorded optically by exciting jRGECO. In the microscopic system, a digital micromirror device (DMD) was used to localize the stimulation with blue light onto the spheroid, while recording from the neighboring monolayer calcium concentration changes. In the macroscopic setup, it was confirmed that the small spheroid was able to pace the whole monolayer and that the waves originated from the spheroid—see sequentially excited spatial points in FIG. 9.

The essential benefit of this system is the extremely short time of magnetically-mediated integration of the “spark-cell” spheroids designed for optical pacing and the non-transformed cardiac tissue/monolayers. Compared to integration without magnetic pull-down, this is several times faster.

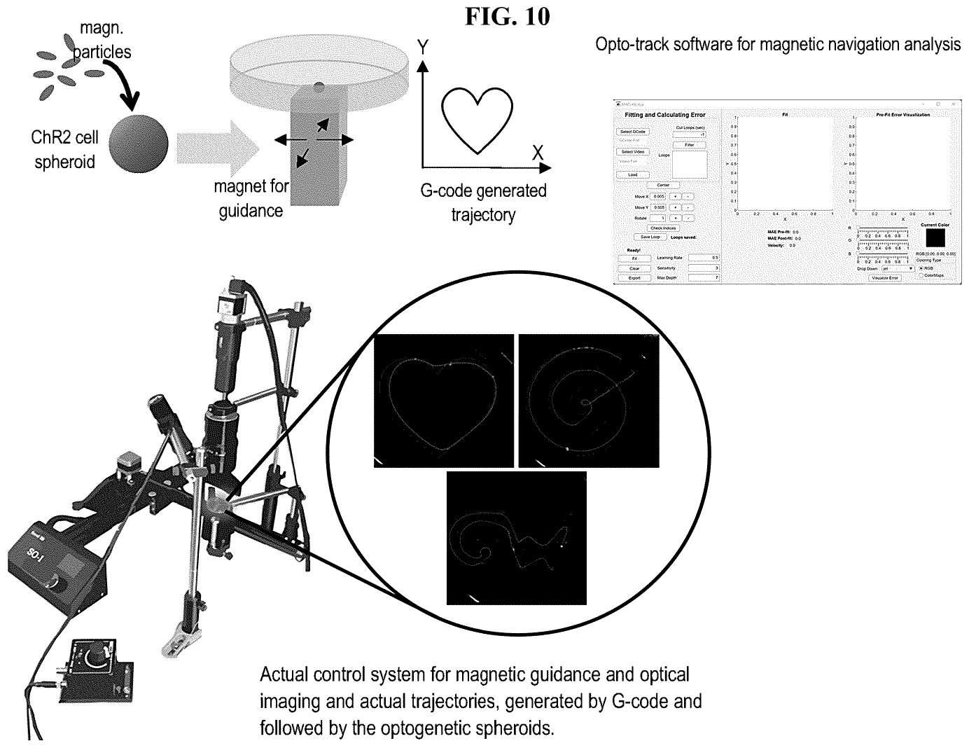

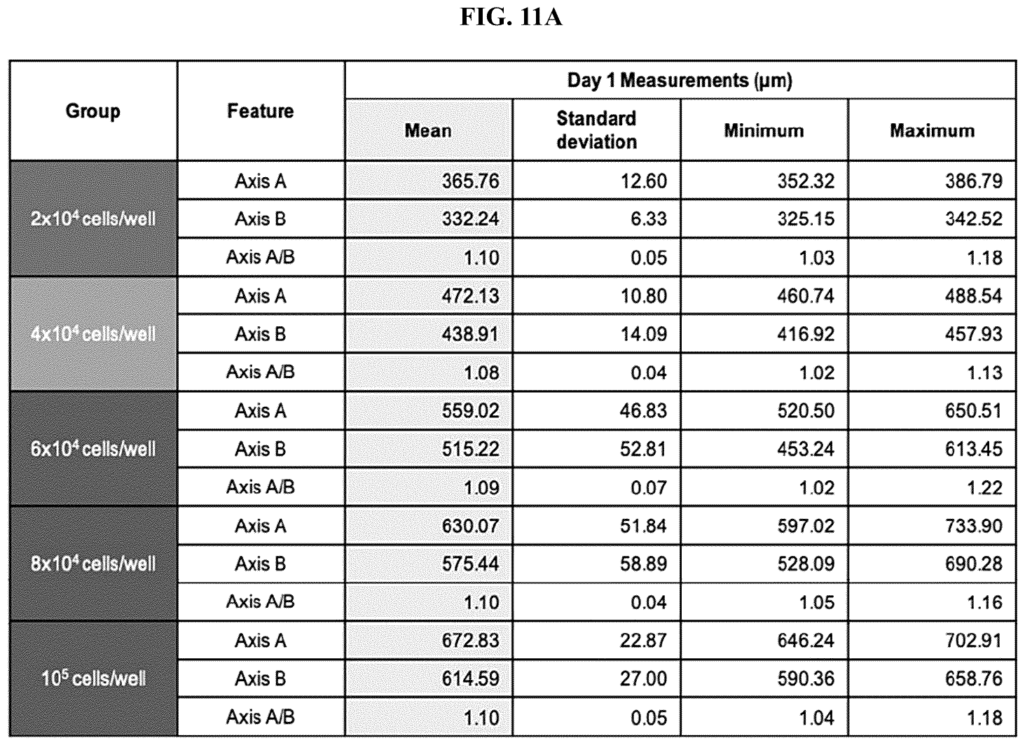

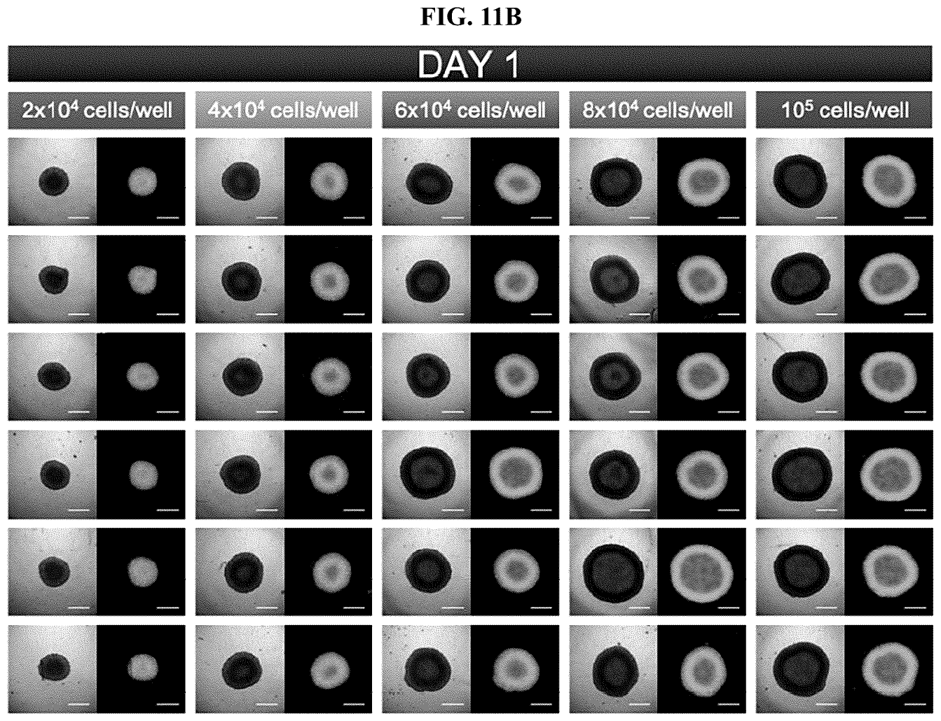

FIG. 10 shows optogenetically- and magnetically-active cell spheroids that were built using both magnetic nanoparticles (0.2 μm diameter, endocytosed) or magnetic microparticles (about 1μm diameter, embedded between the cells). A custom guidance system was built, where a small permanent magnet (3x3 mm tip) was attached onto an x-y-controllable stage. A 35 mm glass-bottom dish with a cell spheroid was positioned above the magnetic guidance system. G-code was used to generate desired trajectories to guide the cell spheroids. A custom-built fluorescence imaging system with large field of view was used to track the spheroids as they are driven along the prescribed trajectory. Custom software (in Matlab) was developed to assess the speed of spheroid guidance, the accuracy of adherence to the prescribed trajectory and various features of the responses.

The simplicity of the magnetic navigation system can be leveraged to guide cell spheroids with engineered properties (optogenetic, sonogenetic, etc.) to a desired location and to deploy them on site for various actuation modalities.

### Discussion and Summary of Examples 1-8

Current methods of high-throughput drug screening, and specifically cardiotoxicity testing, can benefit from optical actuation and sensing methods that offer contactless and scalable interrogation of cardiomyocytes. Optogenetic approaches play a key role in the development of HT all-optical electrophysiology. Some of the limitations associated with (opto)genetic modification of cells include potential interference with their innate functional responses and the time needed for the genetic modification to produce the protein of interest.

In the exemplary methods disclosed herein, “spark-cell” spheroids—usable as a “reagent” that eventually can be stored, transported and deployed on site to confer optical pacing of cardiac tissue—were biomanufactured. Such “spark-cell” spheroids were amenable to automation and could be handled robotically. When deposited onto human iPSC-CM syncytia, they created a spatially localized pacing site. Exemplary data herein showed that this approach may offer faster integration for acute pacing experiments (as early as 2 hours post-deposition in some cases) compared to direct genetic modification of the cardiomyocytes. The mechanism of optical pacing herein is based on the TCU approach and, without being bound by theory, likely involves the formation of close contact and gap junction mediated ion currents between the spheroid and the responding cardiomyocyte syncytia. Further, exemplary methods herein demonstrate a magnetically-mediated integration of the “spark-cell” spheroids designed for optical pacing and the non-transformed cardiac tissue/monolayers at a rate several times faster than that used in the filed currently.

In summary, data disclosed herein demonstrate a scalable, cost-effective method with a cryopreservable reagent to achieve contactless optical stimulation of cardiac cell constructs without genetically modifying the myocytes, that can be integrated in a robotics-amenable workflow for high-throughput drug testing. The approaches disclosed herein can facilitate the assembly of modular designer tissues with space-patterned pacemakers in three-dimensional. Approaches disclosed herein can also find translational applications in the pharmaceutical industry for acute and chronic drug testing. Additionally, the methodologies disclosed herein can also be useful for aiding the long-term maturation of human iPSC-CMs via optical pacing for regenerative purposes.

All the COMPOSITIONS and METHODS disclosed and claimed herein may be made and executed without undue experimentation in light of the present disclosure. While the COMPOSITIONS and METHODS have been described in terms of preferred embodiments, it will be apparent to those of skill in the art that variation may be applied to the COMPOSITIONS and METHODS and in the steps or in the sequence of steps of the METHODS described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A cell-stimulating device, comprising:
a tissue structure comprising:
a plurality of biological cell bodies or non-biological cell bodies; and
disposed in the plurality of cell bodies at least one of
(i) an entity capable of translating one or more external stimuli into a local electric field change, or
(ii) a combination of a first entity capable of translating one or more external stimuli into a local electric field change and a second entity capable of converting photons having a first energy to photons having a second energy.

2. The cell-stimulating device of claim 1, wherein the plurality of cell bodies comprises 100 to 10,000 cell bodies.

3. The cell-stimulating device of claim 1, wherein the tissue structure has a diameter of between 0.15 mm and 1 mm.

4. The cell-stimulating device of claim 1, wherein the one or more external stimuli comprise electrical stimulation, optical stimulation, mechanical stimulation, or magnetic stimulation.

5. (canceled)

6. The cell-stimulating device of claim 4, wherein the one or more external stimuli is a combination of (i) magnetic stimulation and (ii) electrical stimulation, optical stimulation, or mechanical stimulation.

7. The cell-stimulating device of claim 6, wherein the entity disposed in the cell bodies is a magnetic particle.

8. The cell-stimulating device of claim 7, wherein the magnetic particle comprises a magnetic nanoparticle, a magnetic microparticle, or a combination thereof

9. (canceled)

10. The cell-stimulating device of claim 4, wherein the one or more external stimuli is electrical stimulation.

11. The cell-stimulating device of claim 10, wherein the entity disposed in the cell bodies is an ion-channel capable of causing electrical spiking.

12. (canceled)

13. The cell stimulating device of claim 4, wherein the one or more external stimuli is optical stimulation.

14. The cell stimulation device of claim 13, wherein the entity disposed in the cell bodies is a light-gated ion channel or a light-sensitive nanoparticle.

15. (canceled)

16. (canceled)

17. The cell stimulation device of claim 4, wherein the one or more external stimuli is mechanical stimulation.

18. The cell stimulation device of claim 17, wherein the entity disposed in the cell bodies is a mechanosensitive ion channel.

19. (canceled)

20. The cell stimulation device of claim 17, wherein the mechanical stimulation is delivered by ultrasound, fluid flow or pressure puffs.

21. The cell-stimulating device of claim 1, wherein the second entity is configured to emit photons in the absence of external photons and wherein the emitted photons activate the first entity to induce a local electric field change.

22-45. (canceled)

46. A method of preparing a tissue structure capable of growing into the cell-stimulating device of claim 1, the method comprising:
growing a tissue structure on an ultra-low adhesion microplate until it has a diameter of 100 μm to 300 μm;
placing the tissue structure into a cryovial containing a freezing medium, wherein the freezing medium comprises 10% dimethyl sulfoxide (DMSO), 30% Dulbecco's Modified Eagle's Medium (DMEM), and 60% Fetal Bovine Serum (FBS);
freezing the cryovial containing the tissue structure at a controlled rate of 1 degree Celsius per minute overnight in a negative 80 degrees Celsius freezer; and
transferring the cryovial from the negative 80 degrees Celsius freezer to liquid nitrogen.

47. The method of claim 46, further comprising placing the cryovial in a 37 degrees Celsius water bath.

48. The method of claim 47, further comprising mechanically removing ice crystals within 5 minutes of placing the cryovial in the water bath.

49. The method of claim 48, further comprising transferring the tissue structure to a culture medium comprising 98% Dulbecco's Modified Eagle's Medium (DMEM) and 2% Fetal Bovine Serum (FBS), and transferring the tissue structure and the culture medium to an ultra-low-adhesion microplate.

50. The method of claim 49, further comprising culturing the tissue structure for at least an additional 4 days after thawing.

51-60. (canceled)


SEQUENCE LISTING (4 sequences)

SEQ ID NO: 1 - DNA, 22 bp, synthetic construct
  misc_feature 1..22: note = artificial/synthetic
  source 1..22: mol_type = other DNA
ggtggtactcaacagccttatt

SEQ ID NO: 2 - DNA, 22 bp, synthetic construct
  misc_feature 1..22: note = artificial/synthetic
  source 1..22: mol_type = other DNA
accaacatgcacctctcttatc

SEQ ID NO: 3 - DNA, 21 bp, synthetic construct
  misc_feature 1..21: note = artificial/synthetic
  source 1..21: mol_type = other DNA
ggagcgagatccctccaaaat

SEQ ID NO: 4 - DNA, 23 bp, synthetic construct
  misc_feature 1..23: note = artificial/synthetic
  source 1..23: mol_type = other DNA
ggctgttgtcatacttctcatgg